SEQUENCE LISTING (12 sequences)

SEQ ID NO: 1 - DNA, 15 bp, Artificial
  oligonucleotide of invented sequence for use in method for creation of a multiply labeled polymer; eighth base is a cytosine residue containing a primary amine (which is subsequently labeled  with a succinimydal ester containing fluorescent dye);
acgggtccatatctt

SEQ ID NO: 2 - DNA, 14 bp, Artificial
  oligonucleotide of invented sequence for use in method for creation of a multiply labeled polymer
gacccgtaagatat

SEQ ID NO: 3 - DNA, 38 bp, Artificial
  oligonucleotide of invented sequence for use in method for creation of a multiply labeled polymer
acgggtcggcgggacagaagacgcgcagtgagtcggcc

SEQ ID NO: 4 - DNA, 38 bp, Artificial
  oligonucleotide of invented sequence for use in method for creation of a multiply labeled polymer
ggcgggacagaagacgcgcagtgagtcggccatatctt

SEQ ID NO: 5 - DNA, 38 bp, Artificial
  oligonucleotide of invented sequence for use in method for creation of a multiply labeled polymer
gctttttggcgggacagaagacgcgcagtgagtcggcc

SEQ ID NO: 6 - DNA, 14 bp, Artificial
  oligonucleotide of invented sequence for use in method for creation of a multiply labeled polymer
aaaaacgaagatat

SEQ ID NO: 7 - DNA, 38 bp, Artificial
  oligonucleotide of invented sequence for use in method for creation of a multiply labeled polymer
ggcgggacagaagacgcgcagtgagtcggcctttttcg

SEQ ID NO: 8 - DNA, 14 bp, Artificial
  oligonucleotide of invented sequence for use in method for creation of a multiply labeled polymer
gacccgtgcaaaaa

SEQ ID NO: 9 - DNA, 19 bp, Artificial
  oligonucleotide of invented sequence for use in method for creation of a multiply labeled polymer
gacccgtcaaatctacgag

SEQ ID NO: 10 - DNA, 20 bp, artificial
  oligonucleotide of invented sequence for use in method for creation of a multiply labeled polymer
tcacatacgactcaagatat

SEQ ID NO: 11 - DNA, 46 bp, artificial
  oligonucleotide of invented sequence for use in method for creation of a multiply labeled polymer
acgggtcctccactaccgtcttggtttcacatacgactcatatctt

SEQ ID NO: 12 - DNA, 31 bp, Artificial
  oligonucleotide of invented sequence for use in method for creation of a multiply labeled polymer
gagtcgtatgtgaaaccaagacggtagtgga

(19) United States
(12) Patent Application Publication
(10) Pub. No.: US 2006/0160098 A1
(43) Pub. Date: Jul. 20, 2006

(54) Polymeric label molecules

(76) Inventors: S. Arieh Zak, New Rochelle, NY (US); James M. Kadushin, Gilbertsville, PA (US); Robert C. Getts, Collegville, PA (US); Lori Getts, Huntington Valley, PA (US)

Correspondence Address: LAW OFFICE OF MORRIS E. COHEN, 1122 CONEY ISLAND AVENUE, SUITE 217, BROOKLYN, NY, 11230, US

(21) Appl. No.: 11/008,559

(22) Filed: Dec. 9, 2004

Related U.S. Application Data
(63) Continuation of application No. PCT/US03/18768, filed on Jun. 12, 2003.
(60) Provisional application No. 60/388,196, filed on Jun. 12, 2002.

(51) Int. Cl. C12Q 1/68; C12N 15/87

(52) U.S. Cl. 435/6; 435/455

(57) ABSTRACT

A method of constructing a highly labeled linear polymeric molecule for use in any desired application, and linear polymeric molecules of such structure. The polymeric molecule is a nucleic acid constructed from a large number of one or more types of monomeric oligonucleotide units that are attached together to form an extended strand. Within each polymer, at least one type of monomeric unit is provided which is bound to or designed to bind to a label moiety, providing a polymer with a large number of repeat sequences designed for labeling purposes, resulting in extremely effective signal carrying molecule of considerable versatility.

### RELATED APPLICATIONS

The present application is a continuation of PCT Application Serial No. PCT/US03/18768 filed 12 Jun. 2003 (pending), which claims the priority of U.S. Provisional Application Ser. No. 60/388,196 filed Jun. 12, 2002, all of which are fully incorporated herein by reference.

### FIELD OF THE INVENTION

The present invention relates to the synthesis and use of polymeric label molecules.

### BACKGROUND OF THE INVENTION

Molecules containing multiple labels or attachment sites for label moieties are known in the art, including nucleic acid based and other synthetic dendritic structures, hydrocarbon polymers, proteins and other types of molecules. These multiple labeled molecules are bound to primary or secondary targets through the use of hydrogen bond base pair binding (nucleic acid molecules), antibody-antigen interaction, biotin-avidin binding and other common systems.

Typically, nucleic acid probe molecules consist of labeled oligonucleotides, directly labeled cDNA or RNA “runoff” molecules, PCR amplified DNA probes or similar materials. Labeled oligonucleotide probes are readily available and provide efficient kinetics; generally, however, oligonucleotides contain only a single label moiety per probe and offer poor sensitivity.

Directly labeled cDNA or RNA “runoff” probe molecules are significantly larger than oligonucleotide probes and contain multiple labels per molecule incorporated during an enzymatic or similar process. The number of labels per probe is variable and is dependent on the length of the probe, the base composition and the incorporation procedure (enzyme type and concentration, label concentration, efficiency of incorporation, etc.). Hybridization effectiveness may also be affected by the presence of bulky label molecules physically interfering with complementary base pairing. Additionally, unwanted probe molecules are often labeled by enzymes that demonstrate poor specificity for incorporating labels moieties into the target molecules of choice.

Due to the unreliable nature of direct label incorporation and related technologies, there exists a need to provide a labeling product that delivers multiple labels to a target analyte that will not interfere with the normal process of hybridization. Furthermore, there is a need for a product which can be created using a manufacturing process which is cost effective, reproducible and allows for large scale production.

### SUMMARY OF THE INVENTION

It is an object of the present invention to provide a polymeric labeling molecule for use in desired applications.

It is a further object of the present invention to provide a polymeric oligonucleotide labeling molecule containing multiple label moieties.

It is a further object of the present invention to provide an oligonucleotide molecule containing a large number of repeat sequences, wherein each repeat sequence contains or is designed to bind at least one label moiety.

In accordance with the invention, a highly labeled polymeric molecule is provided for use in any desired applications including, but not limited to, applications of biochemical, molecular biological medical, or environmental interest, such as assays, diagnostic tests, reagent kits and so forth. In preferred embodiments the polymeric molecule is a nucleic acid constructed from a large number of one or more types of monomeric oligonucleotide units that are attached together to form an extended strand. These monomers may be reacted with each other to form a highly extendable polymeric strand, as discussed below. The polymer is provided to serve as a label molecule, in other words, to serve as a molecule which generates a detectable signal. Therefore, further to the methods of the invention, rapid, efficient and cost effective synthesis is provided of highly extended polymers which serve as highly effective labels carrying large numbers of label moieties.

Within each polymer, at least one type of monomeric unit is provided which serves as a labeling monomer. Preferably, the labeling monomers are suitably designed oligonucleotides. The term labeling monomer includes both “labeled monomers” and “label-binding monomers”. Labeled monomers include monomers that are designed to include one or more label moieties therein. Label-binding monomers includes monomers that are designed to bind or carry one or more suitable label moieties, and also includes monomers that are capable of binding to other molecules that themselves incorporate or bind labels.

Further to the invention, by repeated and sequential coupling of a population of oligonucleotides including units of labeling monomer therein, a polymeric strand can be synthesized of any desired length and signal strength. These polymeric strands are, therefore, easily and efficiently synthesized as discussed below to deliver very large numbers of label, providing an extremely effective signal carrying molecule of considerable versatility for use in a large variety of potential applications.

Thus, in various preferred embodiments of the invention, a polymeric nucleic acid molecule is provided for use as a labeling molecule, the polymeric nucleic acid being synthesized from a population including one or more types of synthetic or natural oligonucleotide monomers. Within that population, any desired mix can be provided of labeling monomers (whether labeled monomers or label-binding monomers) and non-labeling monomers (monomers which do not incorporate or bind label moieties).

These monomers may be attached together consistent with the invention in any desired manner to form the polymeric structure. In further preferred embodiments, the monomers are polymerized into an extended strand using ligation methods. Preferably, a second population of oligonucleotides are utilized in conjunction with the first population to facilitate the efficiency of ligation of the monomeric units. This second population may be used to provide further labels (e.g. as a second polymeric strand) and/or to provide any desired degree of second strand structure to the final polymer.

In further preferred alternative or additional embodiments, this second population of oligonucleotides is used to facilitate self-assembly of the monomers into the extended polymeric strand. They further serve to increase the efficiency of the ligation between those monomers. Oligonucleotides of this embodiment, also known as “bridging oligonucleotides”, are complementary to 5 prime and 3 prime segments of the monomers in the first population, so each bridging oligonucleotide preferably binds to portions of at least two monomers, aligning them adjacent to each other so that the 5 prime end of one monomer is adjacent to the 3 prime end of another monomer, so that those two monomers can be attached, e.g. by ligation. For example, the bridging oligonucleotide can hybridize to the end of one monomer and to the beginning of another, serving to “bridge” those two monomers together. In this manner, mixture of the bridging oligonucleotides together with the desired labeling monomers in the presence of ligase (and any other desired monomers) results in rapid self assembly of highly labeled polymeric chains.

In yet further preferred embodiments, one or more types of targeting oligonucleotides may be provided in the polymer for attachment of the polymer to further molecules of interest, whether analyte molecules, probes or so forth. Such targeting oligonucleotides are oligonucleotides having a sequence provided for recognition of and binding to a desired molecule of interest (the target).

For example, one or more of the monomers may themselves be designed to serve as targeting oligonucleotides. Thus, a particular monomer within the population for synthesis of the polymer can be provided to serve as a targeting nucleotide alone or as a targeting nucleotide and as a labeling monomer.

In further additional or alternative embodiments, one or more oligonucleotides may be provided which are not monomers (i.e. are not provided for use in growing the polymeric chain in both directions) but rather which are terminating oligonucleotides, i.e. oligonucleotides which are intended to be attached to one or both of the very ends of the polymeric strand. These terminating oligonucleotides may themselves serve as targeting oligonucleotides in addition to or in place of the use of targeting monomers.

Further preferably, the polymer is designed to be linear. Accordingly, an extended polymeric chain can be produced having the desired labeling and targeting properties, those properties being determined by the composition provided to the reaction mixture of labeled monomers, label-binding monomers, non-labeling monomers and/or targeting sequences (whether targeting sequences in the form of targeting monomers and/or terminating oligonucleotides).

Further objects and advantages of the invention will become apparent in conjunction with the detailed disclosure provided herein.

### BRIEF DESCRIPTION OF THE FIGURES

FIG. 1 is a schematic diagram showing synthesis of a labeled polymeric nucleic acid in accordance with a preferred embodiment of the present invention.

FIG. 2a is an illustration of the experimental results of a series of syntheses of polymeric molecules in accordance with the invention, as analyzed using gel electrophoresis. The individual lanes show the variation in polymer sizes at different stoicheometric ratios of 14mer bridging oligonucleotides to 15mer labeled polymeric oligonucleotides.

FIG. 2b is an illustration of the results of the purification of linear polymeric molecules in accordance with the invention.

FIG. 3a is a schematic diagram showing a labeled polymeric nucleic acid (polymer) in accordance with a further embodiment of the present invention, wherein the polymer includes a targeting oligonucleotide at the 5 prime and/or 3 prime end.

FIG. 3b is a schematic diagram showing a method for synthesis of a labeled polymeric nucleic acid in accordance with a further embodiment of the present invention, wherein the polymer includes a terminating targeting oligonucleotide located at the 3 prime end.

FIG. 3c is a schematic diagram showing a method for synthesis of a labeled polymeric nucleic acid in accordance with a further embodiment of the present invention, wherein the polymer includes a terminating targeting oligonucleotide located at the 5 prime end.

FIG. 4a is a schematic diagram showing a method for detection of an analyte nucleic acid by using a labeled polymeric nucleic acid of the present invention hybridized to a capture sequence provided on the nucleic acid analyte.

FIG. 4b is a schematic diagram showing a method for detection of an analyte nucleic acid covalently linked to a labeled polymeric nucleic acid of the present invention.

FIG. 5a is a schematic diagram showing the use of a polymeric nucleic acid hybridized and crosslinked to the arms of a dendritic nucleic acid.

FIG. 5b is a schematic diagram showing the use of a polymeric nucleic acid ligated to the arms of a dendritic nucleic acid.

FIGS. 6 and 7 are schematic diagrams showing further embodiments in which bridging oligonucleotides are used in the synthesis of the linear polymers of the present invention. As shown in the figures, the bridging oligonucleotides align the labeling monomers via binding of nucleotide sequences in the bridging oligonucleotides to complementary nucleotide sequences of oligonucleotides of the first population (e.g. labeling monomers), to position the ends of those oligonucleotides of the first population into a configuration suitable for ligation.

FIG. 6 illustrates an embodiment in which a bridging oligonucleotide is used which binds more than two labeling monomers (six are shown in the figure) to align those labeling monomers into position for ligation into a configuration where the ends of the labeling monomers are positioned for ligation.

FIG. 7 illustrates the sequences of a bridging oligonucleotide in which each bridging oligonucleotide binds to two labelling monomers. The figure illustrates the binding of two bridging oligonucleotides to three labeling monomers to align those labeling monomers.

### DETAILED DESCRIPTION OF THE INVENTION AND THE PREFERRED EMBODIMENTS

In accordance with the present invention, a new polymeric structure is provided for use as a label delivery molecule in any desired context of interest. In accordance with the preferred embodiments of present method, the polymeric molecule is a nucleic acid molecule synthesized using a first population of nucleic acid oligonucleotide molecules, the first population of oligonucleotide molecules being referred to as monomers herein. These monomers of the first population are attached together to form an extended chain via any desired methodology. Preferably, the attachment is via self-assembly, this self-assembly further preferably being conducted via covalent ligation of the monomers in a simple and reproducible enzymatic reaction. In preferred embodiments, the polymeric nucleic acid is preferably linear.

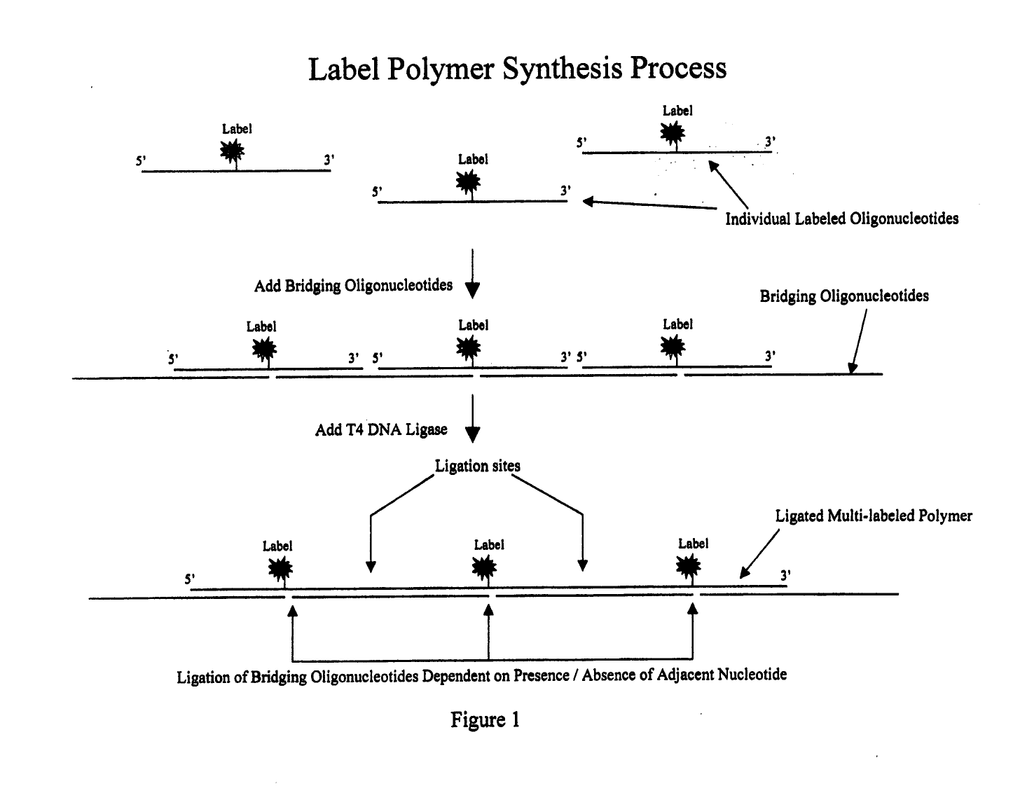

As further illustrated in FIG. 1, in accordance with one series of embodiments of the invention, a first population of oligonucleotide monomers is provided, with the monomers serving as the individual “building blocks” for creation of a polymeric nucleic acid structure. Such oligonucleotide monomers are molecules comprising two or more nucleotides, whether that molecule is naturally occurring or artificially created. In accordance with the invention, the length of the oligonucleotide and its structure can vary, with the particular design of the monomer being tailorable for its intended use. For example, naturally occurring and/or synthetic nucleotide can be used to construct each of the monomers. Examples of naturally occurring nucleotides include, for example, deoxyribonucleotides and ribonucleotides, while examples of synthetic nucleotides include such structures as Locked Nucleic Acid (“LNA”) and Peptide Nucleic Acids (“PNA”). Oligonucleotides suitable for the present invention may be obtained by any desired means, whether synthetically, by cloning or so forth. Numerous known techniques in the art which can be used in conjunction with the present invention are described in Maniatis et al., Molecular Cloning: A Laboratory Manual, 2^{nd} ed., Cold Spring Harbor Laboratory Press, 1989, and Ausubel et al., Current Protocols in Molecular Biology, John Wiley & Sons, Inc., 1998, both of which are fully incorporated herein by reference.

Within the first population of oligonucleotide monomers, at least one subpopulation is provided of “labeling monomers” therein, i.e. monomers that have been synthesized to include one or more label moieties that can generate a detectable signal (“labeled monomers”) or monomers that are capable of binding to a desired label moiety (“label-binding monomers”). As many subpopulations can be provided of labeling monomers as desired, whether one, two, three, four, five, ten, twenty, fifty, or so forth. That first population can also include one or more sub-populations of non-labeling monomers (monomers which do not incorporate or bind label moieties), if desired.

When polymerization is conducted of the oligonucleotide monomers of the first population, those monomers, including any labeling monomers in the first population, are attached to each other producing a polymeric molecule containing a high number of repeat sequences designed to contain or bind to label moieties. One or more label moiety or label moiety binding sequence can be provided in each labeling monomer. Since chains can easily be created having hundreds or thousands of labeling monomers, the resulting final polymeric molecule is an extremely highly labeled signal molecule or signal binding molecule suitable for a variety of applications.

By repeated and sequential coupling of a population of oligonucleotides including units of labeling monomer therein, the polymeric strand which is synthesized can be any desired length and signal strength. Using the present methods, these polymeric strands are easily and efficiently synthesized to deliver very large numbers of label, providing a highly effective signal carrying molecule of considerable versatility.

In various preferred embodiments of the invention this polymeric nucleic acid molecule provided for use as a label molecule is synthesized from a population that includes at least one type of synthetic or natural oligonucleotide monomer in the first population. Mote specifically, within that first population, any desired mix can be provided of labeling monomers (whether labeled monomers or label-binding monomers) and non-labeling monomers (monomers which do not incorporate or bind label moieties).

For example, in one series of embodiments, a population including at least one type of labeling monomer is provided, wherein said labeling monomers are oligonucleotides of the structure 5′-AB3′, wherein AB is a nucleotide sequence such that A corresponds to at least one nucleotide and B corresponds to at least one oligonucleotide; wherein at least one nucleotide in said nucleotide sequence AB incorporates a label or is provided for having a label bound thereto; and wherein said labeling monomers comprises a 5 prime phosphate or being capable of being 5 prime phosphorylated, said labeling monomers further comprising a 3 prime hydroxyl or being capable of receiving a 3 prime hydroxyl.

In further embodiments of the invention, the population includes at least one type of labeling monomer, wherein the labeling monomer is a labeled monomer comprising the structure 5′-ALB-3′; wherein A is at least one nucleotide and contains a 5 prime phosphate or is capable of being 5 prime phosphorylated; wherein B is at least one nucleotide containing a 3 prime hydroxyl or capable of receiving a 3 prime hydroxyl as discussed above. A and B are nucleotides or nucleotide sequences of any type, and L is at least one nucleotide incorporating a label or having a label bound thereto (i.e. a moiety that produces a detectable signal).

Polymerization of the label-binding and/or labeling monomers can be conducted to produce an extremely extended strand carrying a large number of labeled monomers and/or label-binding monomers therein. Thus, the population utilized can be comprised entirely of label-binding labeling monomer, or entirely of labeled monomer.

Or, some mix can be used in the first population of one or more types of label-binding monomer, and/or one or more types of labeled monomer, and/or one or more types of non-labeling monomers. For example, in one embodiment, the population of monomers may include at least two different types of monomers in the initial population, including a first monomer of the structure 5′-AB3′ and a second monomer of the structure 5′-CD-3′. A (and C) are each at least one nucleotide in length and each contain a 5 prime phosphate or are capable of being 5 prime phosphorylated, and B and D are each at least one nucleotide in length containing a 3 prime hydroxyl end or capable of having a hydroxyl group attached thereto. In these embodiments, either the first monomer or the second monomer or both are labeling monomers. In other words, A and/or B and/or C and/or D incorporate a label or are capable of binding a label thereto. Furthermore, in a first set of these embodiments, the sequences A, B, C, and D are not all identical sequences, such that at least one of these sequences differs from the others. In yet further embodiments, A, B, C, and D are all different sequences.

In another such example, at least two types of monomers can be provided in the first population, wherein the monomers are of the structure 5′-AL₁B-3′ and 5′-CL₂D-3′, wherein A is at least one nucleotide and contains a 5 prime phosphate or is capable of being 5 prime phosphorylated; wherein B is at least one nucleotide containing a 3 prime hydroxyl or capable of having a hydroxyl group attached thereto, wherein A and B are nucleotides or nucleotide sequences of any type, and wherein L₁ and L₂ are each at least one nucleotide incorporating a label or having a label bound thereto. L₁ and L₂ can be the same or different sequences, and can include the same or different labels. In a first set of these embodiments, the sequences A, B, C, and D are not all identical sequences, such that at least one of these sequences differs from the others. In yet further embodiments, A, B, C, and D are all different sequences.

Likewise, numerous other configurations of one, two or more types of monomers can be utilized in accordance with the invention. In one preferred set of embodiments, a homogenous population of monomers is provided, i.e. the first population of oligonucleotides includes labeling monomers all of a single sequence. Since all of the monomers within the population are labeling monomers in this embodiment, every monomer of the polymeric chain incorporates or can bind label to maximize the signal delivered. Alternately, the particular composition of the polymeric nucleic acid can be tailored to the needs of the application of interest, with a population of two or more types of monomers utilized, as discussed above.

Any type of label can be used consistent with the invention, with conventional labeling methods known in the art being suitable. The term “label” is used herein in a broad sense to refer to agents that are capable of providing a detectable signal either directly or through interaction with one or more additional members of a signal producing system. Numerous labels may be used consistent with the invention, including, for example, fluorescent labels, chemiluminescent labels, enzymatic labels, and inorganic labels. The label is one that preferably does not provide a variable signal but instead provides a constant and reproducible signal over a given period of time.

Labels that are directly detectable include, but are not limited to, labels detectable by fluorescent detection, chromogenic detection, and chemiluminescent detection, or radioactive labels. Examples include fluorescent labels such as fluorescein, rhodamine, resorufin, and derivatives thereof, coumarins (such as hydroxycoumarin), BODIPY, cyanine dyes (e.g. from Amersham Pharmacia), Alexa dyes (e.g. from Molecular Probes, Inc.), fluorescent dye phosphoramidites, or so forth; and radioactive isotopes, such as 32 S, 32 P, 3 H, etc. Or, marker enzymes such as alkaline phosphatase (AP), beta-galactosidase, or horseradish peroxidase can be used which are detected using a chromogenic substrate. In the case of AP, for example, detection can be effected using 5-bromo-4chloro-3-indolyl phosphate or a nitroblue tetrazolium salt Alternatively, fluorescence resonance energy transfer may also be measured, as described in Cardullo, Nonradiative Fluorescence Resonance Energy Transfer in “Nonradioactive Labeling and Detection of Biomolecules”, C. Kessler, ed., Springer-Verlag, New York, 1992, pp. 414-423, which is fully incorporated herein by reference. Inorganic labels can be used, such as collodial gold particles or ferritin. Detection of labels of colloidal gold particles is described in Van de Plas and Leunissen, Colloidal Gold as a Marker in Molecular Biology: The Use of Ultra-Small Gold Particles, in “Nonradioactive Labeling and Detection of Biomolecules”, C. Kessler, ed., Springer-Verlag, New York, 1992, pp. 116-126, which is fully incorporated herein by reference.

Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. For example, biotin can be used as a binding ligand with avidin or streptavidin as the receptor. One or more labels may be conjugated to the avidin or streptavidin, such as 5(6)-Carboxyfluorescein-N-hydroxysuccinimide ester (FLUOS), 7-amino-4methyl-coumarin-3-acetic acid-N′-hydroxysuccinimide ester (AMCA, activated) and fluorescein isothiocyanate (FITC) which are available from Boehringer Mannheim of Indianapolis, Ind. Methods for fluorescently labeling proteins with fluorescent labels, and methods for detection of the fluorescent labels, are described in Howard, G., Labeling Proteins with Fluorochromes, in “Methods in Nonradioactive Detection,”, G. Howard, Ed., Appleton and Lange, Norwalk, Conn. 1993, pp. 39-68, which is fully incorporated herein by reference. A variety of commercially available labeled streptavidin and avidin molecules are also available, including, for example, streptavidin-gold, streptavidin-fluorochrome, streptavidin-AMCA, streptavidin-fluorescein, streptavidin-phycoerythrin (STPE), streptavidin-sulforhodamine 101, avidin-FITC and avidin-Texas red®, which are commercially available from Boehringer Mannheim, Indianapolis, Ind.

In the preferred embodiments of the invention, the labeled monomers of the population are labeled using a fluorescent dye. Preferably, cyanine dyes such as Cy3 or Cy5 is utilized, although any other suitable fluorescent dye may likewise be employed. One preferred source for such fluorescently labeled monomers is TriLink BioTechnologies, Inc. of San Diego, Calif. However, such fluorescently labeled monomers can be obtained from other sources if desired, or synthesized using methods well known in the art.

Similarly, any desired sequence can be used for the individual monomer, which acts as the subunit or building block for creation of the labeled polymer. Several preferred synthetic sequences have been developed that been found to be of particular use in conjunction with the invention, and are further discussed in the examples below. All of these new sequences have limited or no secondary structure, and all are believed to be abiotic based on the results of typical BLAST searches in Genbank.

The polymer of the invention is preferably created via a coupling together of the individual monomeric subunits. This coupling can be accomplished via any desired process, but is preferably effected via a self-assembly process using ligation of the monomeric units to produce a polymeric structure of the desired length. Alternately, the synthesis of the polymer could be accomplished using a polymerase on a suitably designed template, or by amplification of a sample of linear polymer using known amplification methods, or so forth.

One preferred method for creation of the polymeric structure is shown in FIG. 1. As shown therein, a process is provided wherein a first population of individual oligonucleotide monomers are ligated together to form a polymer of the desired length. Although the example of DNA oligonucleotides is provided for illustrative purposes in the figure, such illustration is not meant to be limiting, as any nucleic acid can be utilized, whether sequences of DNA, RNA, LNA (“Locked Nucleic Acid”), PNA (“Peptide Nucleic Acid”), or so forth, although certain oligonucleotides (such as PNA) will require attachment techniques other than enzymatic ligation which is the preferred embodiment

Self-assembly of monomeric units which are oligonucleotides is the preferred embodiment of the invention as further discussed below. In alternate embodiments, however, other labeled monomeric units can be used, such as sequences of amino acids, linked protein sequences produced via antibody-antigen type interactions, or so forth. Once a population of monomers is provided, those monomers are coupled to form an extended polymeric chain. Any desired means known in the art for assembly of a monomeric unit into a polymeric structure via covalent or non-covalent means can be used consistent with the principles of the invention. For example, oligonucleotide monomers may be assembled using known techniques of molecular biology into an extended nucleic acid strand. Or, labeled amino acid molecules may be assembled into a suitable strand of protein.

In the preferred embodiment, oligonucleotide monomers are assembled using ligation, whether enzymatic or chemical. Preferably, enzymatic ligation is utilized. Techniques of enzymatic ligation of pairs of oligonucleotides are common in the art and utilize well known enzymes called ligases that attach adjacent oligonucleotides to form a continuous oligonucleotide strand. Such methods are generally described for example, in Sambrook, et al., Molecular Cloning: A Laboratory Manual, 2nd Ed., Cold Spring Harbor Laboratory, New York, 1989, which is fully incorporated herein by reference. Representative well characterized and suitable ligases include, but are not limited to, T4 ligase, T7 ligase, Tth ligase, Taq ligase and E. coli DNA ligase, and RNA ligases.

As is known, enzymatic ligation reactions are generally performed in a buffer solution which maintain the pH of the solution at a suitable level for the particular reaction. Other parameters such as temperature, can likewise be adjusted as known. Likewise, the ligation can be conducted in the presence of additives which promote the reaction, whether phosphate transfer agents such as ATP, sulfhydryl reagents, including DTT and 2-mercaptoethanol, and divalent cations such as Mg+2 salts. Similarly, the use of volume excluding agents such as polyethylene glycols (PEG) may be advantageous in promoting ligations, and/or inclusion of up to 200 mM NaCl may also be useful for promoting the ligations. When using the novel synthetic sequences disclosed below designed for use with the present invention, those sequences have been particularly designed to minimize secondary structure. However, using other sequences, single stranded DNA binding proteins can be added to the oligonucleotide ligation reactions to relax any secondary structure in the template strand, thus allowing the complementary oligonucleotides to bind and ligate, improving the reaction's efficiency. For example, E. coli single stranded binding protein (Promega, Madison, Wis. or Amersham/USB) and/or T4 Gene 32 protein (Boehringer Mannheim, Indianapolis, Ind.) can be used.

If desired, the ligase can be a thermostable ligase, in which case thermal cycling techniques are possible. Thermal cycling with a thermostable ligase is useful in various applications, including, for example, methods of amplifying nucleic acids in a manner analogous to the polymerase chain reaction, but using oligomers and a ligase in place of dNTPs and a polymerase. As an alternative to enzymatic ligation, chemical ligation can also be used consistent with the invention. See e.g., K D. James, A. D. Ellington, Chemistry & Biology, 4,595,605, (1997); N-cyanoimidazole: T. Li, K. C. Nicalaou, Nature, 369, 218-221 (1994); EDAC: D. Sievers, G. Von Kiedrowski, Nature, 369, 221-224 (1994), all of which are fully incorporated herein by reference. For example, chemical ligation may be used in conjunction with synthetic oligonucleotides which are not capable of being ligated enzymatically.

Ligation can be conducted with pairs of oligonucleotides having blunt ends, or having an overhang, the latter being preferred. In certain cases, it is believed that a minimum number of nucleotide bases must be presented to particular ligases. (See e.g., C. E. Pritchard and E. M. Southern, Nuc. Acids Res., 25, 3403-3407 (1997), which is fully incorporated herein by reference, discussing a minimum length of about 6-8 bases for reasonable efficiency). In accordance with the invention, ligation is conducted with any number of bases that will result in attachment of the adjacent oligonucleotides, depending on the particular ligase, whether one or two bases, or more. To obtain moderate efficiency, however, at least 4 bases is generally preferred for each oligonucleotide. For optimal efficiency, at least seven bases is preferred in the preferred embodiments of the examples which use T4 DNA Ligase.

The preferred length or range of lengths of each monomer will therefore be influenced by the number of nucleotides needed for optimal functioning of the ligase in question. Thus, some (or preferably all) of the oligonucleotide monomers can be constructed to be of a size which provides a desired level of efficiency in conjunction with a particular ligase. Similarly, the type of label desired will also influence the minimum length of monomer needed, as well as influencing the maximum number of possible labels per monomer. For example, label spacing is often critical to the appropriate function of many types of label moieties, including: 1) fluorescent dyes that demonstrate auto-quenching when spaced too closely; 2) attachment moieties (such as biotin and digoxigenin) that attract other labeling devices that are bulky and may be sterically hindered; 3) enzymes used to generate signal that require adequate spacing for steric or substrate processing reasons; and others. Thus, when using fluorescent dyes such as the cyanine dyes, Cy5 requires a spacing of approximately 6 nucleotides between labels to avoid quenching of the signal, and Cy3 requires a spacing of 9 bases. Radioactive labels, on the other hand, usually have minimal or no spacing requirements.

Thus, in one of the preferred embodiments of the invention, a 15mer oligonucleotide monomer or larger is preferred. The use of a minimum of 15 oligonucleotides in length provides a minimum of 7 bases on each side, since at least 6-7 bases (preferably 7) are required to optimally ligate with T4 DNA Ligase. In further preferred embodiments, the middle of that monomer is being a fluorescent dye such as Cy5 or Cy3. Thus, 14 bases are provided between the dyes, which is more than sufficient to avoid quenching effects. These parameters, however, are merely exemplary of one preferred embodiment. The length of the oligonucleotide can, of course, can generally be designed to suit the desired context, depending on such factors as the ligase to be used, the labels to be used, the number of labels that will be included per monomer (e.g. one label per individual monomer, or 2-5 labels, or 6-10, 10-20, 21-100, etc.) or so forth.

Further in accordance with the preferred embodiment, the reaction mixture includes a second population of oligonucleotides in the form of complementary “bridging molecules”. Each of these bridging molecules are used to link and align two or more monomers of the first population, the bridging molecules serving as “scaffolds” for the subsequent ligation. Furthermore, each complementary bridging oligonucleotide, not only spans and links at least two monomers, it also provides a double stranded portion on either side of the ligation site between those monomers, this double stranded segment being a condition required for certain ligase reactions, such as that conducted for example by T4 DNA Ligase mediated DNA to DNA ligation.

As shown in the preferred embodiment of FIG. 1, each of the oligonucleotide bridging molecules in the second population will hybridize to two monomers from the first population in such a manner so as to link those two monomers, spanning the ligation site between them. For example, the complementary bridging molecules can each be provided with a first sequence of nucleic acid complementary to the beginning of each monomer, and be provided with a second segment complementary to the end of each monomer, thereby “bridging” the two monomers together across the intended site of ligation.

In alternate embodiments, the bridging oligonucleotide can be provided to bridge more than two monomers, if desired. For example, a long bridge molecule can be used which recruits three, four, five or more monomers. In these embodiments, the “bridge” molecule only overlaps the monomers at the edges of the bridging molecule, with one or more monomers hybridizing to the bridging molecule (e.g. at the center) without overlap. Similarly, even those monomers hybridizing at the ends of the bridging molecule (whether using two or more monomers per bridge), do not need to overhang since ligation can be conducted of blunt ends. However, the use of overhangs at the ends is preferred, as it is generally believed to provide more efficient ligation.

In the event that the first population includes multiple types of monomers to be ligated, those different types of monomers will preferably all have the same sequence at the 5′ end and 3′ end to allow them to be aligned by one population of bridging molecules. Alternately, multiple bridging molecules can be used when there are multiple types of monomers present in the first population.

The bridging monomers also provide a second strand to the polymer. This second strand may be continuous or discontinuous, as desired. For example, the bridging molecules may be sufficiently long such that the bridging molecules will abut each other when they bridge together two monomers (allowing the bridging molecules to also be ligated by the ligase); or, the bridging molecules can be shorter than the monomers such that there is are gaps on the second strand between adjacent monomers producing a polymer having both double stranded and single stranded segments.

In some applications, a double stranded final polymer will be preferred, in which case the bridging oligonucleotides can be sufficiently long to be ligated together, with the final double stranded molecule being intact for use in the application. In other applications, a single stranded final polymer will be preferable, in which case the two strands can be separated, or one strand can be degraded, using known methods. In yet further embodiments, the final polymer may have both single and double stranded segments, the single stranded segments being located in the spaces between between non-abutting bridging molecules.

In the various embodiments, the bridging molecules may be used merely to bridge together pairs of monomers to facilitate their self-assembly. In further embodiments, however, the bridging molecules can themselves include labels or be designed to carry labels thereon. For example, the bridging oligonucleotides may also be monomers analogous to the monomers of the first population described above. In this embodiment, the oligonucleotides of the second population also include labeling nucleotides therein in the same manner as described with reference to the first population. Therefore, when the first and second population both include labeling monomers, those two populations serve to both to bridge each other and to generate a polymer with labels on both strands.

In accordance with the invention, the polymeric molecules synthesized by the method of the invention contain at least 2 synthetic oligonucleotide molecules containing label moieties. However, more preferably, polymers of at least 5, 10, 20, 50, 100, 500, 1000, or thousands, or more monomers can easily be synthesized consistent with the invention. Thus, polymers containing at least 5, 10, 20, 50, 100, 500, 1000, or thousands, or more label moieties can be easily generated.

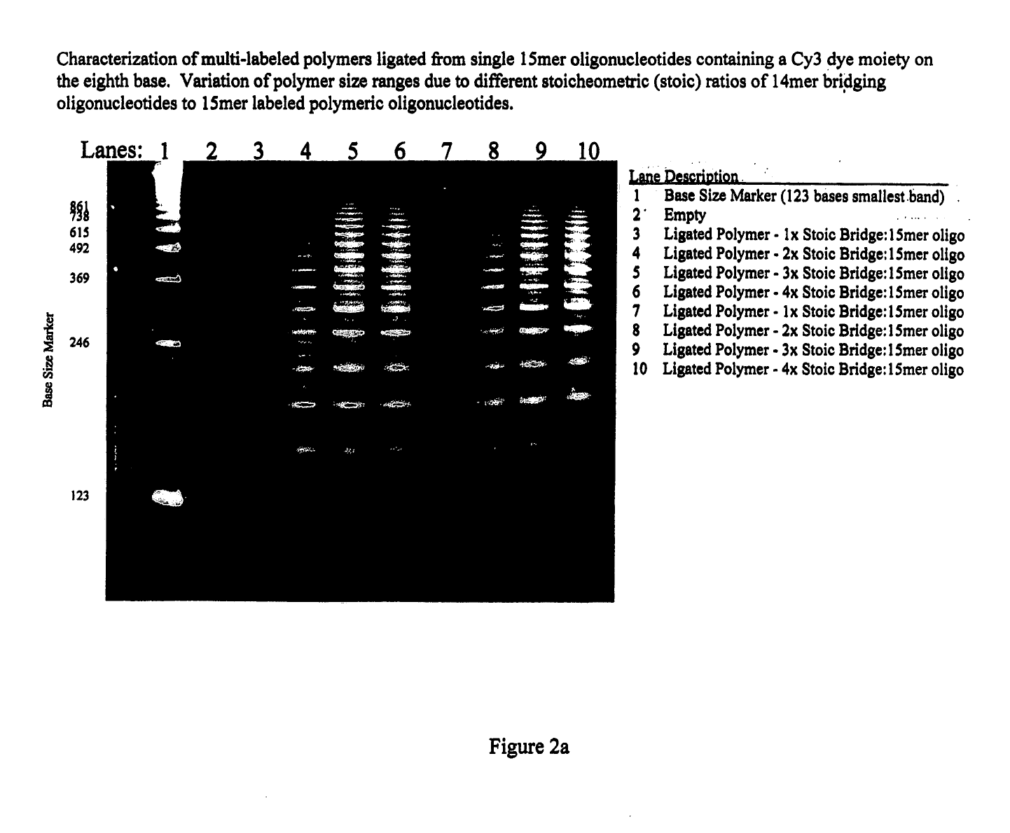

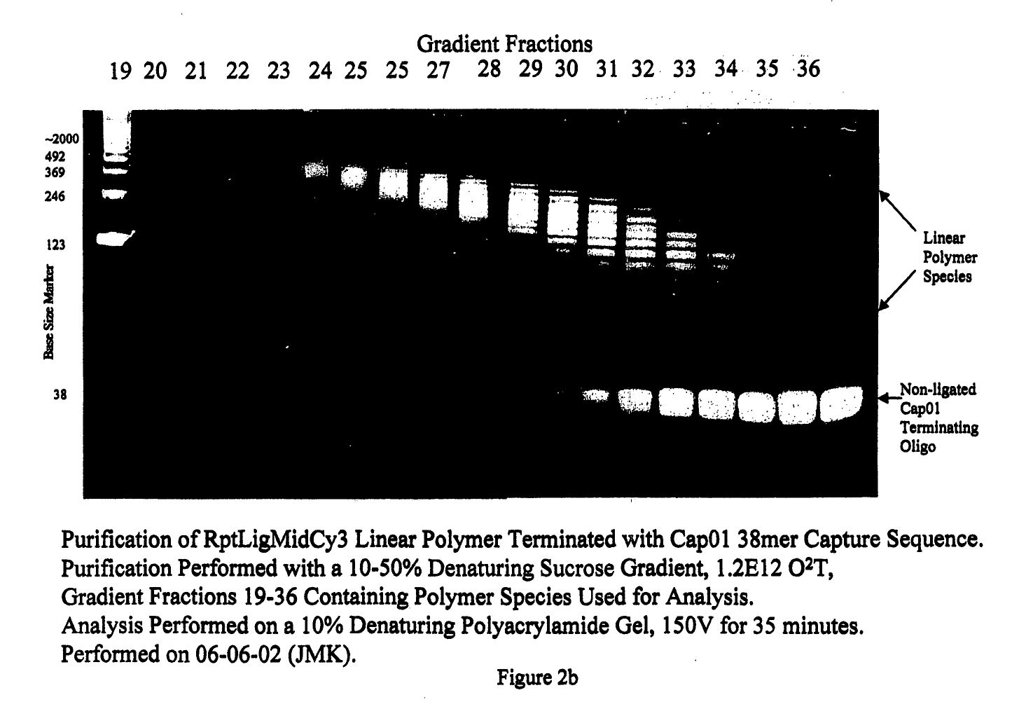

In general the synthesis of polymeric nucleic acids using the preferred methods of ligating monomers described herein will generate a population of polymers of varying lengths. When the monomers are are all labeled, the lengths of these polymers will correspond to the number of label moieties contained within the polymeric molecule. FIG. 2a, for example, illustrates the experimental results from a series of syntheses of polymeric molecules, and shows the variation of ranges in polymer size at several differing ratios of bridging molecule to oligonucleotide. In the preferred embodiments shown in the figure, the 15mer oligonucleotides used in the experiment were each labeled with a Cy3 fluorescent dye moiety on the eighth base, with a base marker being provided in lane 1 and differing ratios of a 14mer bridging oligonucleotide to the 15mer oligonucleotides used as monomers being provided in lanes 3 through 10. Lanes 3 and 7 show the variation in polymer sizes produced using a 1:1 ratio of bridging molecule to oligonucleotide; Lanes 4 and 8 show the use of a 2:1 ratio of bridging molecule to oligonucleotide; Lanes 5 and 9 show a 3:1 ratio of bridging molecule to oligonucleotide; and Lanes 6 and 10 show a 4:1 ratio of bridging molecule to oligonucleotide.

For a given application, shorter or longer linear polymers may be desired based on the needs of the assay or so forth, in view of factors such as as kinetics, etc. The desired length of that linear polymeric molecule produced can, therefore, be tailored by adjusting the reaction conditions and by purifying molecules of the desired length.

Whether short or long, these polymers are extremely packed with label moieties. Furthermore, these highly labeled molecules can be produced without using radioactivity or generating radioactive waste. Moreover, the present invention provides the significant advantage that synthesis of these highly labeled polymeric nucleic acids is easily achieved using a simple mixture of: at least one species of ligatable oligonucleotide which serves as a monomer, a complementary bridging oligonucleotide, a suitable ligase (e.g. a DNA or RNA ligase), ligation buffer, and ATP. In addition, reactions can be typically be completed in a few minutes to several hours. The conditions and concentrations of reactants can be modified as desired to determine the average length of the ligation products.

Consistent with the invention, suitable reagents for producing the linear polymers of the present invention can be provided individually, or in kits. For example, suitable oligonucleotide monomers and/or complementary bridging oligonucleotides can be provided in kits for producing a linear polymer or as individual reagents. Such kits can further include any other desired reagents disclosed in the present application, such as ligase, ATP, ligation buffer, terminating oligonucleotides, and so forth.

Further in accordance with the invention, the polymeric labeling molecule can be attached to a further molecule by any desired means. In preferred embodiments, the polymeric label molecules are rendered targetable by attaching targeting structures to either the 3 prime or 5 prime ends of the polymer, or both of those ends. Such targeting structures can be attached via a variety of reactions including blunt end or overhanging ligation, base extension of the 3 prime end with terminal d-transferase (Tdt), and other known processes. Alternately, one or more of the monomers can themselves be directly or indirectly attached to a further molecule, or to a moiety attached to a further molecule.

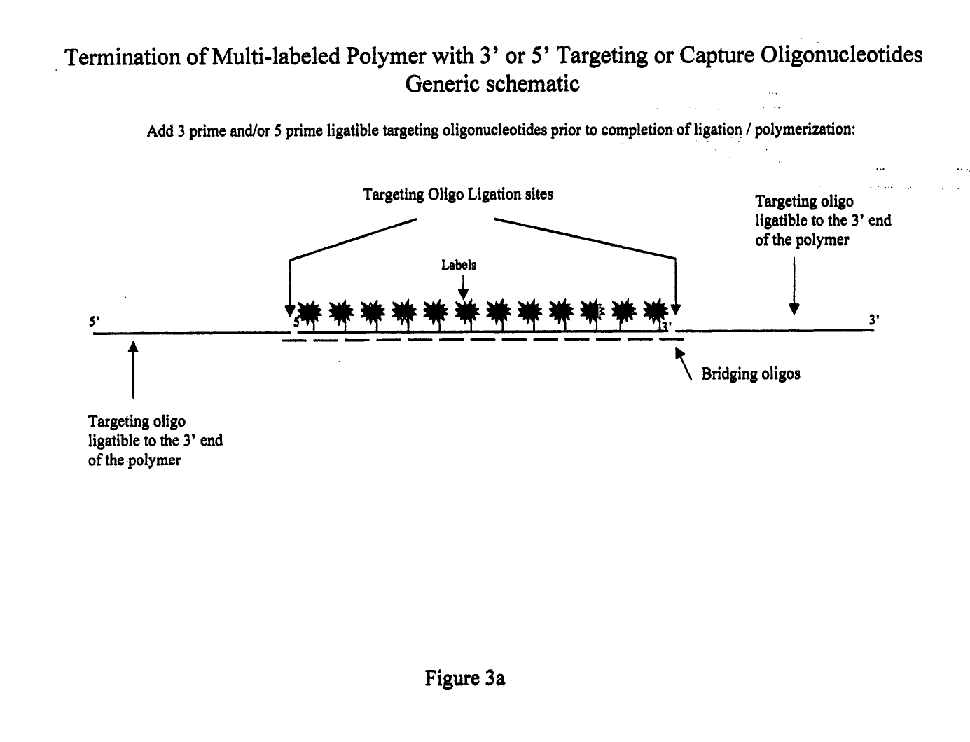

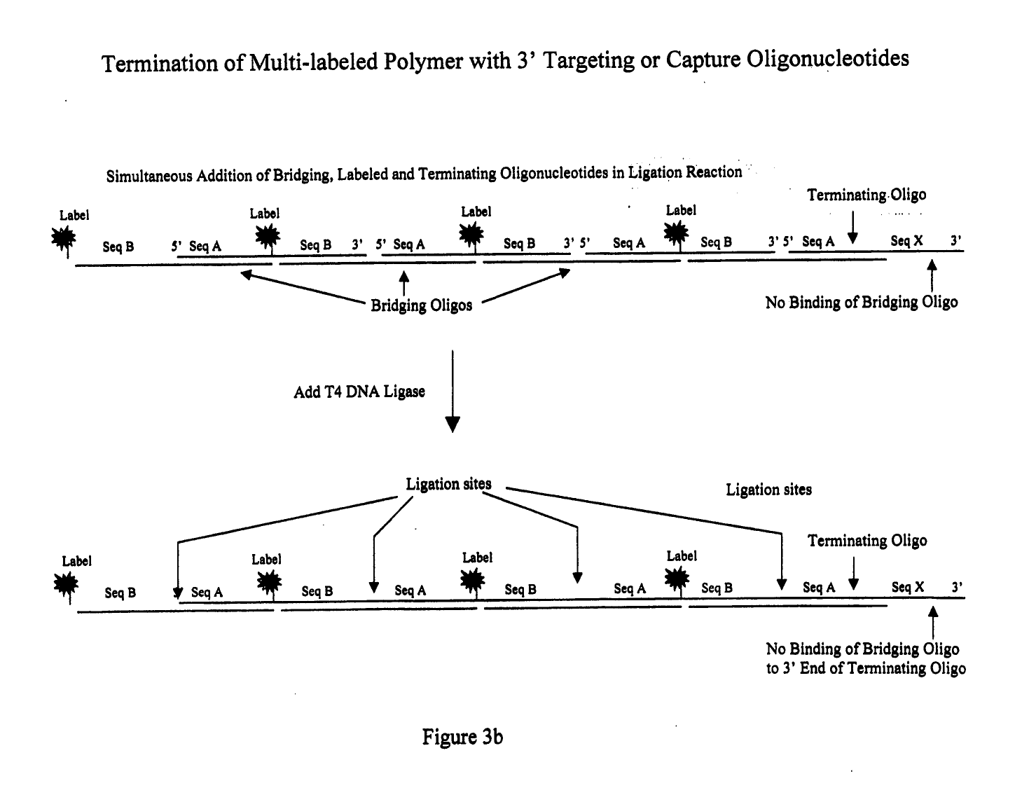

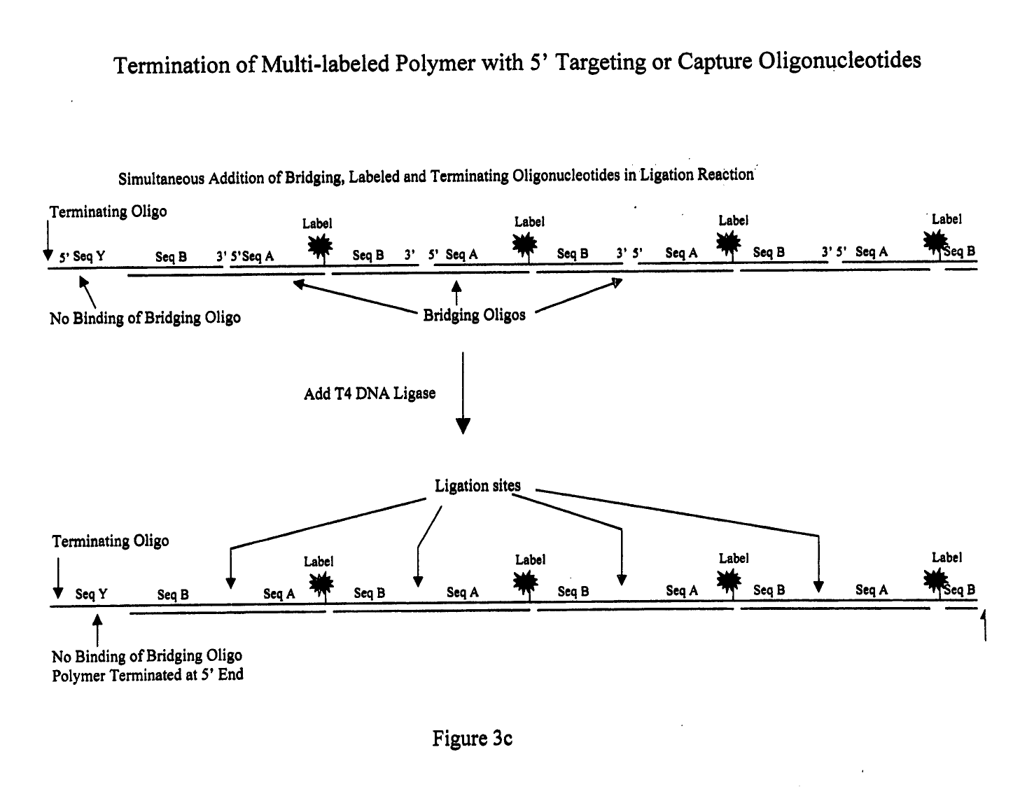

As illustrations of the use of terminating oligonucleotides for targeting purposes, FIGS. 3a, 3b and 3c illustrate the attachment of 3 prime and/or 5 prime targeting or capture oligonucleotide molecules to a polymeric molecule via a standard overhanging DNA to DNA ligation reaction. As shown in FIG. 3a, for example, a targeting oligonucleotide can be attached to the 3 prime and/or 5 prime end of the oligonucleotide via ligation. The terminating targeting oligonucleotide can be added prior to, during, or after completion of the polymerization of the monomers via ligation, resulting in further ligation of the targeting oligonucleotide to the growing polymeric strand.

This targeting oligonucleotide is preferably chosen to be complementary to a sequence on a second molecule, allowing capture of the polymer via hybridization of the complementary strands. For example, the targeting oligonucleotide can be a complement to a sequence on an analyte molecule, a dendrimer, a further polymeric oligonucleotide of the present invention, or so forth. This sequence on the second molecule is used to capture the labeled polymer, and is therefore referred to as the “capture sequence” herein.

It has been found, however, that the addition of a targeting or terminating oligonucleotide sequence (i.e. one different than the labeled monomer), to a simultaneous polymeric ligation reaction serves as a partial inhibitor to the ligation reaction, resulting in a reduction of the average size of the polymers produced. Inhibition has been found to be dependent on the concentration of the added targeting nucleotide. Therefore, when a targeting oligonucleotide is added to terminate the polymer, it is desirable to limit the concentration of that terminating oligonucleotide to a concentration that provides the maximum size and yield of labeled polymer molecules containing the targeting oligonucleotide. By way of definition, the term terminating oligonucleotide herein refers to any oligonucleotide other than the monomeric unit added onto the 5 prime or 3 prime end of the polymer. Such a terminating oligonucleotide can be added before the polymerization reaction, simultaneously during the polymerization reaction, or subsequent thereto. FIG. 3a illustrates an embodiment in which the targeting oligonucleotide is added to both the 5 prime and 3 prime ends of the polymeric molecule.

As shown in FIG. 3b, in an alternate embodiment of the invention, the targeting oligonucleotide may be added to one end of the polymer only. For example, the targeting oligonucleotide can be attached to the 3 prime end only, as shown in the figure. In the embodiment of FIG. 3b, the bridging oligonucleotides, labeled oligonucleotides (monomers) and terminating oligonucleotides can all be simultaneously added to the reaction mixture with a suitable ligase (e.g. T4 DNA ligase, or so forth). Alternatively, the terminating oligonucleotides can be added prior to or after the ligation. The terminating oligonucleotide includes a 5 prime end (“Sequence A”) which is complementary to the overhanging end of the bridging oligonucleotide at the 3 prime end of the polymer.

However, the terminating oligonucleotide is further provided with a 3 prime end (“Sequence X”) which is not complementary to the bridging oligonucleotide and which will not hybridize thereto. As a result of the inability of the 3 prime end (“Sequence X”) of the terminating oligonucleotide to hybridize to the bridging oligonucleotide, no hybridization of terminating oligonucleotide to bridging oligonucleotide will occur at the 3 prime end of the polymeric molecule, and polymerization will cease at that end. Subsequent ligation of the monomeric units to each other, and ligation of the monomer at the 3 prime end to the terminating oligonucleotide, will result in a polymer having a monomeric sequence at the 5 prime end of the polymer molecule, and having a terminating oligonucleotide sequence at the 3 prime end of the polymer molecule.

The same principles can used to hybridize the terminating (termination) oligonucleotide to only the 5′ end of the polymeric molecule, as shown in FIG. 3c. In such an embodiment, a non-complementary sequence to the bridging oligonucleotide (“Sequence Y”), is provided at the 5 prime end of the terminating oligonucleotide, with a complementary sequence to the bridging molecule (“Sequence B”) being provided at the 3 prime end of that terminating oligonucleotide (also referred to as the termination oligonucleotide). Addition of monomeric units will therefore cease at the 5 prime end of the growing molecule, with polymerization ceasing at that 5 prime end.

Likewise, two different types of terminating oligonucleotides can also be used. By using both types of oligonucleotides discussed above with reference to FIGS. 3b and 3c, one type of terminating oligonucleotide can be hybridized to the 5 prime end of the polymer, with the other to the 3 prime end of the polymer.

Once the polymers have been formed, various methods may be used to purify different populations of the polymeric molecule based on size, number of labels and other parameters. Typically, size exclusion resins may be used to exclude smaller fractions of molecules. Continuous or discontinuous gradients are useful for collection of fractions containing largest to smallest molecules. Polyacrylamide gel purification, HPLC, FPLC, affinity chromatography, affinity beads, or other common manual or automated systems are also useful for separating molecules of varying size. Functionally, different sized polymeric molecules of the invention may be used in different applications, e.g. smaller molecules can be more suitable for certain in-situ hybridization assays requiting hybridization of the labeled polymer to less accessable cellular structures; whereas, longer polymers with more labels can be more suitable for use on microarrays for better amplification of signal.

The labeled polymeric molecules may have potential uses in detection of target molecules, including nucleic acids, proteins and peptides, carbohydrates, lipids and others. Typical assay applications include fluorescent and chemiluminescent microarrays and macroarrays, in-situ hybridization utilizing fluorescent and enzymatic labels, flow cytometry, microtiter plate ELISA and hybridization assays, and other applications.

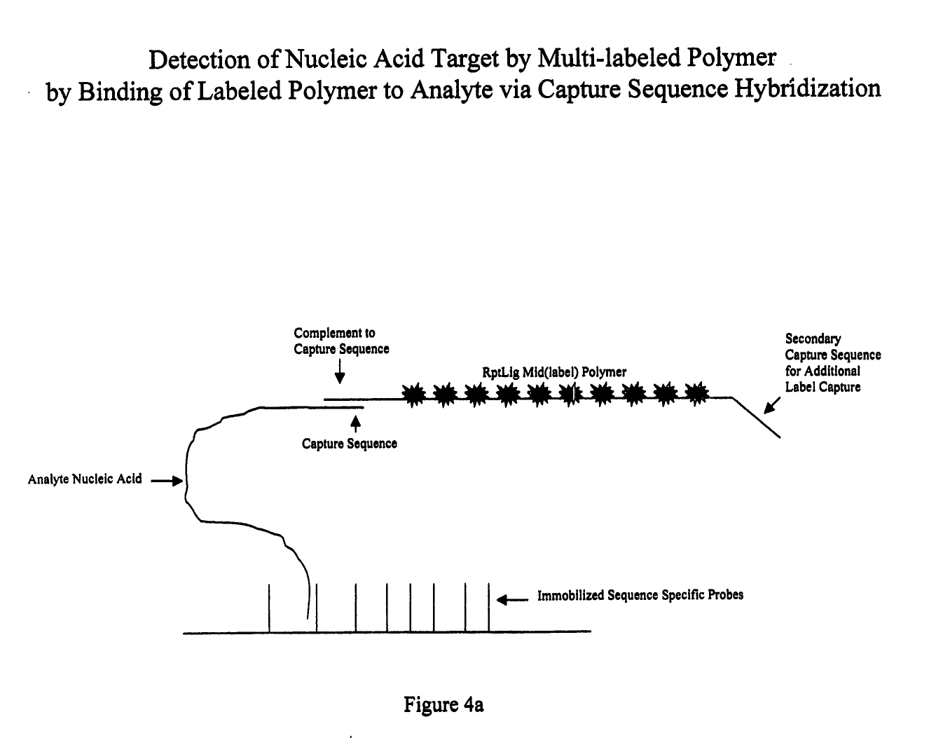

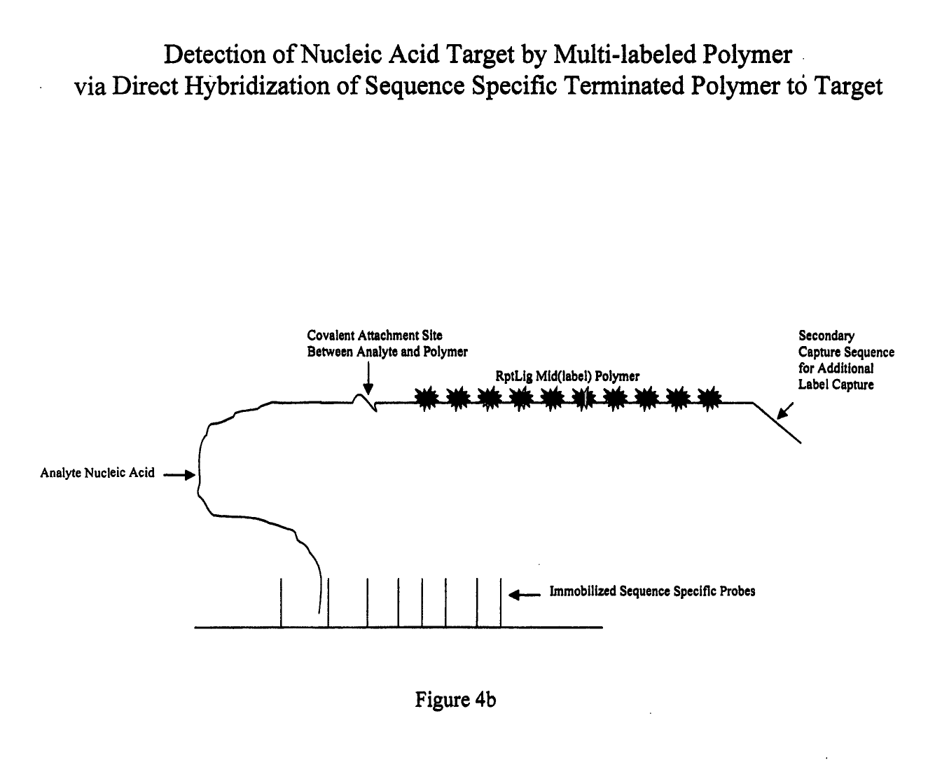

In various embodiments of the invention, the polymer of the present invention is attached to a further molecule via one or more of the monomeric units, and/or a terminating oligonucleotide, and/or a moiety on either one or more monomers or terminating oligonucleotides. In one series of preferred embodiments, for example, the terminating oligonucleotide is hybridized to a complementary sequence on another nucleic acid, the complementary sequence being used to “capture” the terminating oligonucleotide and polymer. The complementary sequence is also referred to as the “capture sequence” herein. FIGS. 4a and 4b, for example, illustrate a indirect and direct method, respectively, for binding the polymer to nucleic acid probes, the first example of FIG. 4a being conducted via a capture sequence bridge and the second example of FIG. 4b via a direct coupling of the polymer to the probe.

In FIG. 4a, for example, the polymer is provided with a terminating oligonucleotide at one end which is complementary to a capture sequence on an analyte molecule. The analyte molecule may be any desired molecule of interest In preferred embodiments, the analyte is a molecule of cDNA. For example, molecules of cDNA can be synthesized which include the capture sequence by providing the capture sequence as part of the primer used in reverse transcription process. Further details of such embodiments are disclosed in PCT Application Serial Number PCT/US01/07477 filed Mar. 8, 2001 (Int'l Publication No. WO 01/066555), PCT Application Serial No. PCT/US01/22818 filed Jul. 19, 2001 (Int'l Publication No. WO 02/06511), and PCT Application Serial No. PCT/US01/29589 filed Sep. 20, 2001 (Int'l Publication No. WO 02/033125), PCT Application Serial No. PCT/US02/05022 filed Feb. 20, 2002 (Int'l Publication No. WO 03/012147), PCT Application Serial No. PCT/US02/27799 filed Sep. 3, 2002 (Int'l Publication No. WO 03/020902), all of which are fully incorporated herein by reference. Hybridization of the capture sequence of the analyte molecule to the complementary terminating oligonucleotide captures the labeled polymer, resulting in labeling of the analyte nucleic acid. These analyte nucleic acids can be hybridized to probes of known sequence, e.g. probes immobilized to a solid surface such as a microarray, or membrane, or in-situ hybridization (ISH), or so forth.

Alternately, instead of hybridization of the polymer to the analyte molecule, the polymer can be directly covalently attached to the analyte nucleic acid to label that nucleic acid, as shown in FIG. 4b. This covalent attachment can be conducted before or after hybridization of the analyte nucleic acid to probes, e.g. probes immobilized to a solid support such as a microarray, a membrane, in-situ hybridization (ISH), or so forth, as discussed above.

The polymeric label molecules may also be useful as label moieties attached to a delivery device, i.e. an entity which collects many of the polymers and delivers a multitude of polymers to a target. Such delivery devices, therefore, can act as signal amplification molecules, i.e. molecules which can themselves hold multiple label molecules, and/or can act as targeting systems, Le. systems for attaching to a desired target

Thus, when a signal amplification molecule carries multiple polymers of the present invention, a signal amplification molecule is provided which itself carries signal amplification molecules (i.e the polymer), such that an extremely efficient signal molecule is provided. This can likewise be iterated as many times as useful e.g. to form a complex such that a signal amplification carries a second signal amplication molecule which carries a third signal amplification, and so on, as many times as desired. However, any such design preferably should take into account the kinetics of the final complex.

Various delivery devices or signal amplification molecules can be used consistent with the invention. In preferred embodiments, the delivery device or signal amplification molecule is a dendrimer, whether a dendrimer of nucleic acid or other components (e.g. plastics or so forth). Most commonly, dendrimers are dendritic nucleic acid molecules in the form of complex, highly branched molecules comprised of a plurality of interconnected natural or synthetic monomeric subunits of double-stranded nucleic acid (e.g. DNA or RNA). Such dendritic nucleic acid molecules are described in greater detail in Nilsen et al., Dendritic Nucleic Acid Structures, J. Theor. Biol., 187, 273-284 (1997); in Stears et al., A Novel Sensitive Detection System for High-Density Microarrays Using Dendrimer Technology, Physiol. Genomics, 3: 93-99 (2000); and in various U.S. patents, such as U.S. Pat. Nos. 5,175,270; 5,484,904; 5,487,973; 6,072,043; 6,110,687; and 6,117,631. All of those publications are incorporated herein by reference.

Dendrimers comprise two types of single-stranded hybridization “arms” on the surface which are used to attach two key functionalities. A single dendrimer molecule may have at least one hundred arms of each type on the surface. One type of arm can be used, for example, for attachment of a specific targeting molecule to establish target specificity, with the other being used for attachment of a label or marker such as the polymeric labeling molecule of the present invention. The molecules that determine the target and labeling specificities of the dendrimer are attached either as oligonucleotides or as oligonucleotide conjugates. Using simple DNA labeling, hybridization, and ligation reactions, a dendrimer molecule may be configured to act as a highly labeled, target specific probe.

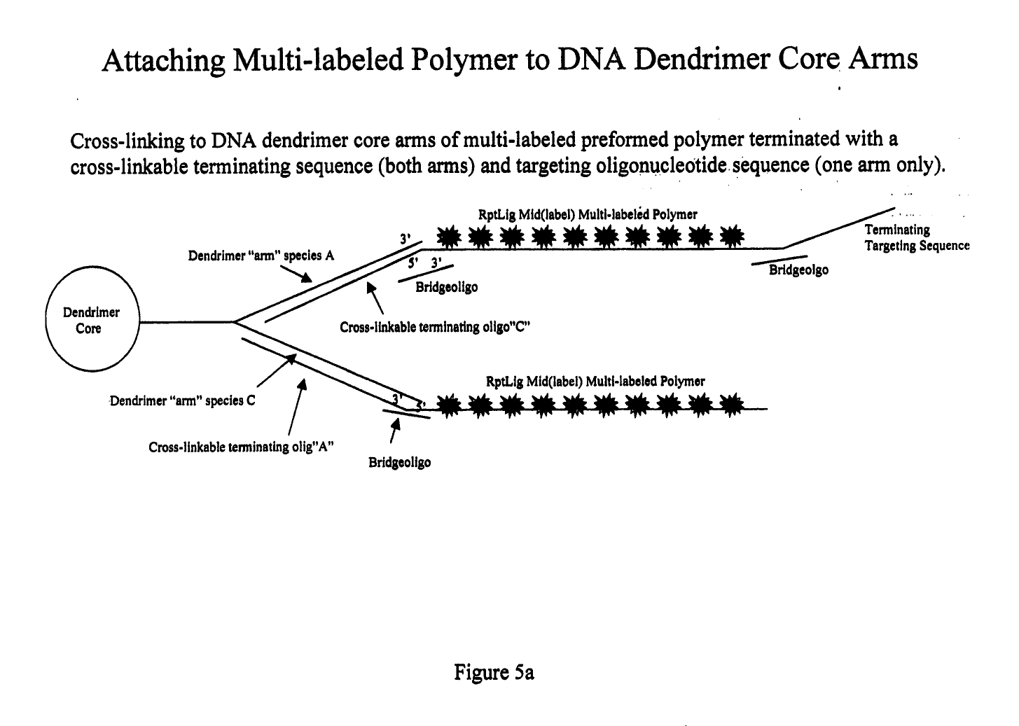

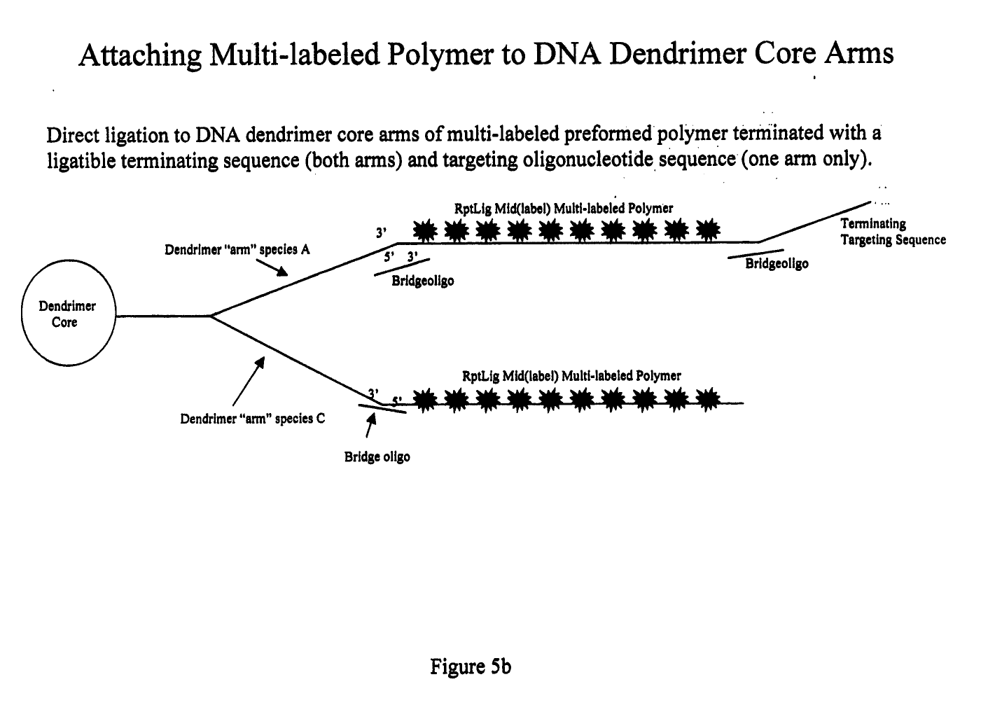

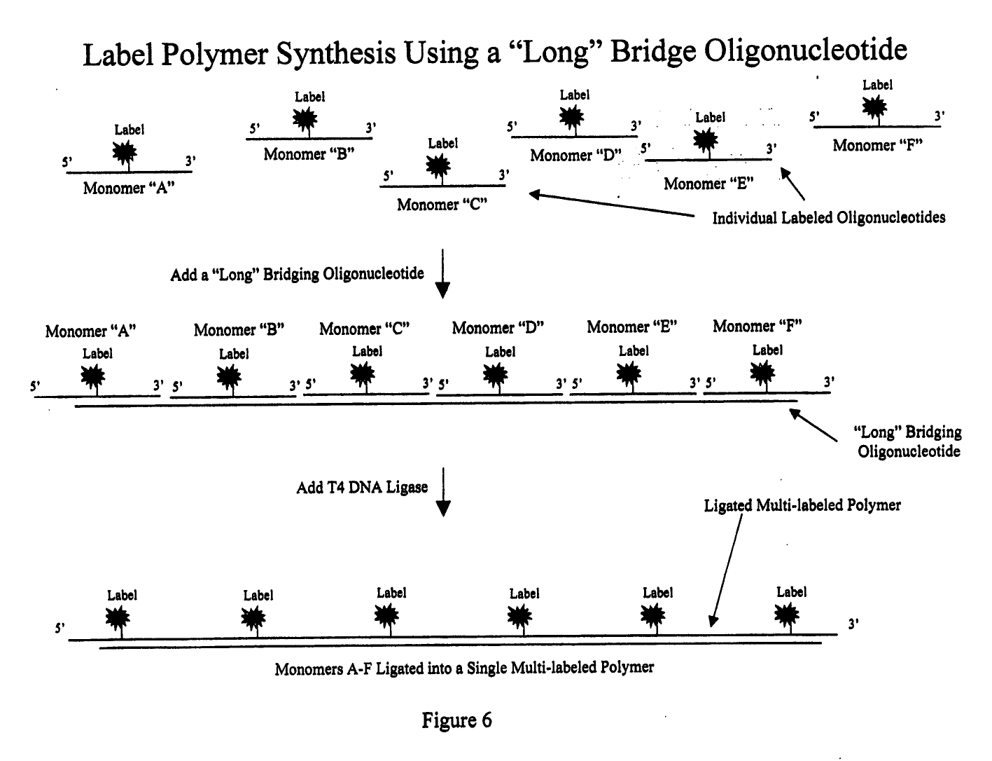

For example, a labeled linear polymer of the present invention can be hybridized to one of the types of arms of the dendrimer, with a target sequence being hybridized to another type of arm of the dendrimer using a capture sequence. Such embodiments are disclosed, for example, in PCT Application Serial Number PCT/US01/07477 filed Mar. 8, 2001 (Int'l Publication No. WO 01/066555), PCT Application Serial No. PCT/US/01/22818 filed Jul. 19, 2001 (Int'l Publication No. WO 02/06511), and PCT Application Serial No. PCT/US01/29589 filed Sep. 20, 2001 (Int'l Publication No. WO 02/033125), PCT Application Serial No. PCT/US02/05022 filed Feb. 20, 2002 (Int'l Publication No. WO 03/012147), PCT Application Serial No. PCT/US02/27799 filed Sep. 3, 2002 (Int'l Publication No. WO 03/020902), all of which are fully incorporated herein by reference. Thus, the polymer nucleic acid of the present invention can be used as the signal molecule referred to in those applications as being attached to a dendrimer arm. Or, the labeled linear polymer can be attached to both types of arms of the dendrimer, yet further enhancing signal strength, as shown in FIG. 5. Various additional embodiments and inventions are also disclosed in those PCT applications, any of which can be used in conjunction with the inventions of the present application, or modified to use the present invention.

The schematics for assembling a dendrimer delivery device is illustrated in further embodiments shown in FIGS. 5a and 5b. As shown in FIG. 5a, for example, a polymer in accordance with the present invention can be attached to the arms of a dendritic molecule. The polymer can be attached to one or more types of arms of the dendrimer, for example, by hybridization of a terminating oligonucleotide on the polymer to a dendrimer arm containing a complementary sequence. By using a terminable oligonucleotide having a crosslinkable sequence, the terminating oligonucleotide that hybridizes to the dendrimer arm can be crosslinked to the dendrimer arm to covalently attach the polymer to the dendrimer. Any suitable crosslinking agent known in the art can be used consistent with the invention.

Preferably, crosslinking agents such as the psoralens can be used, which are linear furocoumarins having the ability to crosslink DNA strands upon photoactivation. See e.g., Glass, U.S. Pat. No. 4,826,967, which is fully incorporated herein by reference. Alternatively, photoactivatable compounds such as nucleoside analogues prepared by linking the phenyl ring (especially at the 7 position) of a coumarin or coumarin analogue to the 1 position of a D-ribose or D-2-deoxytibose molecule can be used. See e.g., Saba, U.S. Pat. No. 5,082,934, which is fully incorporated herein by reference, with the double bond between the 3 and 4 positions of the coumarin ring system is the photoactivatible group that covalently crosslinks to nucleosides in the complementary strand when a probe containing this nucleoside analogue is used in a hybridization assay. Other crosslinking agents can likewise be used consistent with the invention.

Instead of hybridization and crosslinking of the end of the linear polymer to the dendrimer arm, as shown in FIG. 5a, the linear polymers may alternatively be provided with a ligatable terminating sequence on one end. These terminating sequences can be ligated to the dendrimer arms, as shown in FIG. 5b.

Furthermore, in accordance with the methods of FIGS. 5a and 5b, two linear polymers can be utilized. As shown in the figures, a first labeled linear polymer can be attached to one type of dendrimer arm, and is used for signalling purposes. A second linear polymer is also provided and is attached on one end of the polymer to the second type of dendrimer arm. On the second end of this linear polymer, a terminating oligonucleotide is provided having a targeting sequence.

Once non-attached materials are purified out (i.e. to leave only dendrimer with the polymer attached and no free polymer or free terminating oligonucleotides or undesirable contaminants), the targeting sequence can be used to hybridize the linear polymer to a probe. Thus, the entire dendrimer complex with linear polymers attached to its numerous arms is hybridized to the probe, whether a probe is on a microarray, on a blot, or whether other methods are used such as in-situ hybridization (ISH), or so forth. Since the second linear polymer not only includes the targeting sequence, but is also labeled, signal strength is yet further enhanced over the use of a label on one type of arm alone.

In yet further embodiments, linear polymers can be created for attachment to both types of dendrimer arms, wherein both linear polymers include the targeting sequence at the second end of the polymer. Thus, in this embodiment, both arms of the dendrimer are labeled and also have targeting sequences attached for hybridization to the probe, to yet further enhance probe-target sensitivity and specificity.

Additional delivery devices include nucleic acid molecules synthesized enzymatically or chemically produced that include moieties capable of binding one or more linear labeled polymer molecules. This for example, includes but is not limited to:

(1) DNA molecules synthesized by a reverse transcriptase reaction from an RNA template using a nucleotides containing a moiety capable of binding linear polymer molecules. An example of this would include nucleotides labeled with a primary amine capable of chemically binding a linear polymer end containing a succinimidyl ester, sulfhydryl or equivalent compatible crosslinkable moiety.

(2) DNA molecules containing crosslinkable moieties (as above) enzymatically synthesized from the use of DNA polymerases. Examples of this include PCR amplified or similarly produced molecules.

Or, (3) RNA molecules containing crosslinkable moieties (as above) enzymatically synthesized from the use of RNA polymerases. Examples of this include RNA runoff transcripts produced from DNA templates containing T7, T3 or SP6 RNA promoter sequences and the appropriate RNA polymerase enzyme.

Additionally, the present invention is particularly useful in conjunction with microarrays, although it is also applicable to other assay systems. A number of different microarray configurations and methods for their production are well known to those of skill in the art, one of which, for example, is described in Science, 283, 83, 1999, the contents of which is fully incorporated herein by reference. In general, the microarray is a high-speed technology useful for nucleic acid analysis, and includes a plurality of distinct nucleic acid or gene probes (i.e., polynucleotides) distributed spatially, and stably associated with a substantially planar substrate such as a plate of glass, silicon or nylon membrane. The substrate, therefore, is coated with a grid of tiny spot (e.g. of microns in diameter); each spot (i.e. feature) containing millions of copies of a short sequence of DNA or nucleotides; with a computer keeps track of the location of each sequence on the substrate. Labeled target molecules are subsequently applied to those probes, and the array is washed to remove target molecules that have not hybridized. The presence of labeled molecule at a particular location on the array can then be corresponded with the known sequence of probe at that location. Such microarrays have been developed and are used in a range of applications such as analyzing a sample for the presence of gene variations or mutations (i.e. genotyping), or for patterns of gene expression, and allow one to perform the equivalent of thousands of individual “test-tube” experiments carried out in a short period of time. For example, microarray assays may be conducted using the methods of FIG. 4 or 5 of the present invention, or so forth.

Further aspects of the invention will be apparent in conjunction with the following examples. It will be understood that the examples are intended to illustrate various preferred embodiments of the invention, without forming a limitation on the scope of the present invention, as discussed in the disclosure herein and as set forth in the appended claims.

### EXAMPLE 1

### Synthesis of Multiple Labeled Polymer Labeled with Fluorescent Dye

Synthesis of a repeating polymeric DNA molecule containing multiple fluorescent dye labels was accomplished by using the following 15mer synthetic DNA oligonucleotide (synthesized by standard amidite chemistry methods), as the polymerizing component:

(SEQ. ID NO.1)

5′- phos - ACg ggT C C(SE-Cy3) ATA TCT T -3′

The 5 prime end of the oligonucleotide is chemically (or enzymatically) phosphorylated and the eighth base of the oligo is a cytosine residue (C) containing a primary amine, which is subsequently labeled post oligonucleotide synthesis (and pre-polymerization) with a succinimydal ester containing fluorescent dye (in this example, Cyanine dye Cy³) in a standard chemical condensation reaction. This oligonucleotide is also referred to as “RptLigMidCy3”.

A bridging oligonucleotide capable of spanning the seven 5 prime nucleotides and seven 3 prime nucleotides of RptLigMidCy3 was synthesized utilizing standard amidite oligonucleotide synthesis chemistry, the bridging oligonucleotide having the following sequence:

5′- gAC CCg TAA gAT AT -3′(SEQ. ID NO.2)

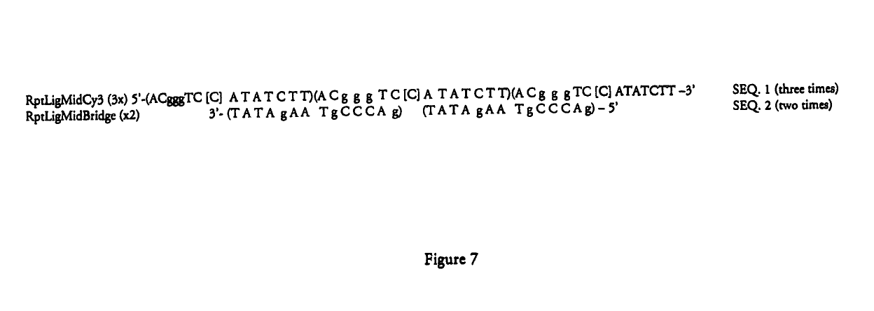

This oligo is also referred to as “RptLigMidBridge”, and hybridizes to the RptLigMidCy3 oligonucleotide as illustrated in FIG. 7.

As shown in FIG. 7, multiples of RptLigMidCy3 oligonucleotides align end to end via the hybridization to the RptLigMidBridge bridging oligonucleotide. (It may be noted that the convention of use of lower case g to refer to Guanine in this and other examples is provided merely to more readily differentiate Guanine residues to the reader from Cytosine signified by upper case C).

Covalent bonds between adjacent RptLigMidCy3 molecules were created through the enzymatic action of T4 DNA ligase, which ligates between adjacent 3 prime and 5 prime (phosphorylated) DNA ends in the presence of a low salt buffer containing divalent cations and adenisine triphosphate (ATP). Using T4 DNA ligase, enzymatic ligation is also contingent on there being a double stranded sequence on either side of the ligation site, with at least three (3) base pairs on each side of the ligation site being required for minimal ligation, and up to seven (7) base pairs on each side of the ligation site required for optimal ligation efficiency. A typical ligation reaction is described below:
A. Combine in a nuclease free polypropylene microfuge tube the following components:

$$\begin{matrix}107.1 uL - RptLigMidCy3 oligonucleotide at 1.0 ugms \text{/} uL ( aqueous ) \\ 280.0 uL - RptLigMidBridge oligonucleotide at 1.0 ugms \text{/} uL ( aqueous ) \\ \begin{matrix}100.0 uL - 10 X Ligation Buffer ( Roche catalog number 1 \; 243 \; 292 ) , \\ comprising 660 mM Tris \text{-} HCl , 50 mM MgCL2 , \\ 50 mM ditriethreotol ( DTT ) , 10 mM ATP\end{matrix} \\ 512.9 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1 U per uL, Roche catalog number 481 220). Mix gently with a pipetter.
F. Incubate at 4-20 C for 30-720 minutes (or longer). Longer incubation at temperatures below 20 C produce slightly more efficient ligation, resulting in somewhat longer concatenation ligation products.
G. Stop the reaction by adding 27.5 uL of 0.5M EDTA (aqueous).
H. Concentrate the volume to ˜100 uL using the Microcon YM-30 Microconcentrators (Millipore) according to the manufacturer's instructions.

The typical result of the above process is a heterogeneous range of sizes of the ligated RptLigMidCy3 polymeric molecules derived from single oligonucleotides, as indicated by denaturing polyacrylamide gel electrophoresis (PAGE) (See e.g., FIG. 2a). Purification of specific size ranges is accomplished by stratification of the polymer species by size using a denaturing 50% formamide/variable sucrose concentration gradient. The gradient is cast into 5 mL polyallomer ultra-centrifuge tubes (Beckman cat # 328874) using the model J5 Gradient Former (Jule Inc., Milford, Conn.) such that the top of the gradient is approximately 10% sucrose and the bottom of the gradient is approximately 50% sucrose, with the increase of density from top to bottom roughly linear. 50-100 uL of the post ligation concentrated RptLigMidCy3 heterogeneous polymer is loaded onto the top of the cast gradient, and the gradient is centrifuged in the Beckman Optima L-70K Ultracentri for 15 hours at 45,000 RPM (omega square T of 1.2E12) using the Sw60 rotor (Beckman). Post centrifugation, the gradient tube is punctured at the bottom and the gradient solution is harvested from the bottom up, dropwise, collecting 2-5 droplet fractions per collection tube. The fractions are analyzed by PAGE and characterized by the range of size for a particular fraction (FIG. 2b). The number of fluors for each fraction is calculated by dividing the average size by 15 (the number of residues per RptLigMidCy3 oligonucleotide). Empirical measurement of the fluor to mass ratio is performed by calculating the mass per volume concentration as derived from standard 260 nm spectroscopy measurement and then measuring the specific fluorescence of a known quantity of mass on the Spex FluoroMax 3 spectrofluorometer instrument (Jobin-Yvon-Horiba Ltd., Japan). Specific fluorescence measurements of the RptLigMidCy3 polymer are performed for excitation/emission wavelengths of 542 nm and 570 nm respectively (derived for Cy3 to avoid Raman water excitation/emission peaks). Average number of functional Cy3 fluors per molecule for any particular fraction is calculated via the use of a standard curve of the independently measured fluorescence of different quantities of a single fluor labeled oligonucleotide.

### EXAMPLE 2

### Synthesis of Multiple Labeled DNA Polymer with Non-Polymeric Oligonucleotides Capable of 3 Prime End or 5 Prime Ligation to the Labeled DNA Polymer in a Simultaneous Ligation Process

Addition of a second population of oligonucleotide sequences to serve as terminating oligonucleotides on either or both ends of the labeled polymers allows for the specific hybridization or binding of the polymeric molecules to a desired sequence. The complementary sequences can be used as capture sequences to hybridize to the complementary terminating sequence as discussed above. Uses of these constructs include direct primary targeting of nucleic acid molecules in a variety of hybridization platforms (including blots, microarrays, in-situ hybridization (ISH) and others). Alternatively or additionally, labeled polymers may provide secondary (or tertiary) labeling of primary targeting molecules by hybridizing the labeled polymers to sequence complementary to the labeled polymer bound terminating sequence. Primary label delivery devices may include DNA dendrimers, plastic or magnetic beads, polymeric molecules containing complementary sequence, or so forth.

It has been found that the addition of a terminating sequence (containing a sequence different than the labeled polymer) to a simultaneous polymeric ligation reaction serves as a partial inhibitor to the ligation reaction, resulting in the reduction of the average size of the polymers dependent on the concentration of the terminating oligonucleotide. Therefore, it is desirable to limit the concentration of the terminating oligonucleotide to a concentration that provides the maximum size and yield of labeled polymer molecules containing the terminating oligonucleotide.

This terminating reaction is similar to the ligation reaction in Example 1, except for the addition of a terminating oligonucleotide of any appropriate sequence containing sequence complementary to the seven 3 prime or 5 prime nucleotides of the bridging oligonucleotide RptLigMidBridge.

In this example, the oligonucleotide RptCap01-5′lig, with the sequence:

5′- phos - ACg ggT Cgg Cgg gAC AgA AgA CgC gCA gTg
AgT Cgg CC -3′

will ligate via its 5 prime end to the RptLigMidCy3 oligonucleotide, or terminating oligonucleotide RptCap01-3′lig, with the sequence:

(SEQ. ID NO.4)

5′- ggC ggg ACA gAA gAC gCg CAg TgA gTC ggC CAT
ATC TT -3′

will ligate via its 3 prime end to the RptLigMidCy3 oligonucleotide. These terminating oligonucleotides contain 5 prime and 3 prime ends (respectively) of seven nucleotides complementary to the RptLigMidBridge bridging oligonucleotide. The terminating oligos in this example are useful for targeting the resulting terminated RptLigMidCy3 polymer, e.g. by hybridizing the terminated polymer to complementary capture sequences found on primers used to synthesize cDNA from RNA in a reverse transcriptase enzymatic reaction. The incorporation of capture sequences into cDNA during the reverse transcription from RNA, and the use of capture sequences and their complements is further discussed, for example, in PCT Application Serial Number PCT/US01/07477 filed Mar. 8, 2001 (Int'l Publication No. WO 01/066555), PCT Application Serial No. PCT/US01/22818 filed Jul. 19, 2001 (Intl Publication No. WO 02/06511), and PCT Application Serial No. PCT/US01/29589 filed Sep. 20, 2001 (Int'l Publication No. WO 02/033125), PCT Application Serial No. PCT/US02/05022 filed Feb. 20, 2002 (Int'l Publication No. WO 03/012147), and PCT Application Serial No. PCT/US02/27799 filed Sep. 3, 2002 (Int'l Publication No. WO 03/020902), all of which are fully incorporated herein by reference.

These terminating molecules, capable of ligating to the 3 prime and 5 prime ends (respectively) of the RptLigMidCy3 oligonucleotide, will not block the opposite end of the RptLigMidCy3 oligonucleotide from continuing to ligate additional RptLigMidCy3 oligonucleotides via ligation onto the appropriate free end, thereby allowing the synthesis of concatenated RptLigMidCy3 labeled oligonucleotides to the non-blocked ends of the polymer. The ligation reaction is performed as follows:
A. Combine in a nuclease free polypropylene microfuge tube the following components:

$$\begin{matrix}107.1 uL - RptLigMidCy3 oligonucleotide at 1.0 ugms \text{/} uL ( aqueous ) \\ 280.0 uL - RptLigMidBridge oligonucleotide at 1.0 ugms \text{/} uL ( aqueous ) \\ 27.1 uL - RptCap01 \text{-} 5 ’ lig or RptCap01 \text{-} 3 ’ lig oligonucleotide at \\ 1.0 ugms \text{/} uL ( aqueous ) \\ 100.0 uL - 10 X Ligation Buffer ( Roche catalog number 1 \; 243 \; 292 ) , \\ 485.8 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1 U per uL, Roche catalog number 481 220). Mix gently with a pipetter.
F. Incubate at 4-20 C for 30-720 minutes (or longer). Longer incubation at temperatures below 20 C produce slightly more efficient ligation, resulting in somewhat longer concatenation ligation products.
G. Stop the reaction by adding 27.5 uL of 0.5M EDTA (aqueous).
H. Concentrate the volume to ˜100 UL using the Microcon YM-30 Microconcentrators (Millipore) according to the manufacturer's instructions.
   Purification of the heterogeneous polymer species resulting from ligation is via denaturing gradient purification (see e.g., Example 1).

### EXAMPLE 3

### Synthesis of Multiple Labeled Polymer with Non-Polymeric Sequence Specific Oligonucleotides Capable of 3 Prime End or 5 Prime Ligation to the Multiple Labeled Polymer in a Non-Simultaneous Pre-Priming Ligation Process

As previously noted in Example 2, simultaneous ligation of the termination oligo to the multiple labeled polymer can demonstrate poor efficiency if the termination oligo is used in excess. An alternative to that method that improves ligation efficiency requires the use of a bridging oligo that uniquely hybridizes to one end of the polymerizing oligonucleotide but not the other end; the other end is designed to specifically bind to the terminating oligonucleotide only, thereby allowing the specific ligation of the terminating oligonucleotide to a single polymerizing oligonucleotide only. This specific ligation reaction is performed as an initial “priming” reaction that creates a population of hybrid terminating and polymerizing oligonucleotide molecules that are then ready for the polymerizing reaction as described in Example 1.

In this example, the oligonucleotide RptCap01-7BO-5′lig, with the sequence:

(SEQ. ID NO.5)

5′- phos - gCT TTT Tgg Cgg gAC AgA AgA CgC gCA gTg
AgT Cgg CC -3′

will ligate via its 5 prime end to RptLigMidCy3, via the use of the bridging oligonucleotide RptBr7BO-3′:

5′-AA AAA Cg A AgA TAT -3′(SEQ. ID NO.6)

or terminating oligonucleotide RptCap01-7BO-3′lig, with the sequence:

(SEQ. ID NO.7)

5′- ggC ggg ACA gAA gAC gCg CAg TgA gTC ggC CTT
TTT Cg -3′

will ligate via its 3 prime end to RptLigMidCy3 oligonucleotide, via the use of the bridging oligonucleotide RptBr7BO-5′:

5′- gAC CCg TgcAA AAA -3′(SEQ. ID NO.8)

A method of the priming ligation is described below:
A. Combine in a nuclease free polypropylene microfuge tube the following components:

$$\begin{matrix}100.0 uL - RptLigMidCy3 oligonucleotide at 1.0 ugms \text{/} uL ( aqueous ) \\ 100.0 uL - RptBr7BO \text{-} 3 ’ or RptBr7BO \text{-} 5 ’ Bridge oligonucleotide at \\ 1.0 ugms \text{/} uL ( aqueous ) \\ 100.0 uL - RptCap01 \text{-} 7 BO \text{-} 5 ’ lig or RptCap01 \text{-} 7 BO \text{-} 3 ’ lig \\ oligonucleotide at 1.0 ugms \text{/} uL ( aqueous ) \\ 100.0 uL - 10 X Ligation Buffer ( Roche catalog number 1 \; 243 \; 292 ) , \\ 600.0 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1 U per uL, Roche catalog number 481 220). Mix gently with a pipetter.
F. Incubate at 20-25 C for 30 minutes.
G. Add the following reagents to the above reaction to continue the RptLigMidCy3 non-specific polymerization:

$$\begin{matrix}1100.0 uL - Priming ligation reaction from above \\ 25.0 uL - RptLigMidCy3 oligonucleotide at 2.0 ugms \text{/} uL ( aqueous ) \\ 150.0 uL - RptLigMidBridge Bridge oligonucleotide at 1.0 ugms \text{/} uL \\ ( aqueous ) \\ 25.0 uL - 10 X Ligation Buffer ( Roche catalog number 1 \; 243 \; 292 ) , \\ 25.0 uL - Nuclease Free Water \\ 1 , 325.0 uL total volume\end{matrix}$$
H. Incubate at 20-25 C for 30 minutes.
I. Repeat step G five (5) additional times.
J. Stop the reaction by adding 61.35 uL of 0.5M EDTA (aqueous).
K Concentrate the volume to ˜100 UL using the Microcon YM-30 Microconcentrators (Millipore) according to the manufacturer's instructions.
   Purification of the heterogeneous polymer species resulting from ligation is via denaturing gradient purification (see Example 1).

### EXAMPLE 4

### Labeling of a DNA Dendrimer with a Fluorescent Polymer by Direct Polymerization on the Dendritic Structure

Labeling of signal amplification molecules such as DNA dendrimers with large quantities of fluorescent or other labels allows for higher signal intensities than what is otherwise achievable through current labeling techniques. This is accomplished by first ligating a single oligonucleotide (capable of further polymerization) to the 3 prime or 5 prime ends of the dendritic structure (“priming oligonucleotide”), followed by cyclic or non-cyclic ligation of the same or different additional polymeric molecules to the “priming oligonucleotide”. This method labels a DNA dendrimer with polymeric multi-labeled DNA strands of the present invention extending from the free ends (“branches” or “arms”) of the dendritic structure. Targeting oligonucleotides may be attached to the dendrimer “arms” via UV crosslinking or ligation prior to or after ligation of the polymeric oligonucleotides; alternatively, targeting oligonucleotides may also be attached to the distal ends of the polymeric multi-labeled strands after ligation of these structures to the dendrimer “arms”.

A typical reaction would include the use of the following oligonucleotides:

(SEQ. ID NO.1)

5′- phos - ACg ggT C C(SE-Cy3) ATA TCT T -3′

the polymeric labeled oligonucleotide previously described as “RptLigMidCy3”.

5′-gAC CCg TCA AAT CTA CgA g -3′(SEQ. ID NO.9)

a “bridging” oligonucdeotide called “c(−)RptLig 5 prime” required to hybridize to the 5 prime end of the RptLitMidCy3 oligonucleotide and to the 3 prime end of the dendrimer “arm”. The “c(−)RptLig 5 prime” oligonucleotide aligns the 5 prime phosphorylated end of the RptLigMidCy3 and the 3 prime end of the dendrimer arm such that ligation will occur between the dendrimer arm and only one RptLigMidCy3 oligonucleotide.

5′- TCA CAT ACg ACT CAA gAT AT -3′(SEQ. ID NO.10)

a “bridging” oligonucleotide called “a(−)RptLig 3 prime” required to hybridize to the 3 prime end of the RptLitMidCy3 oligonucleotide and to the 5 prime end of the dendrimer “arm”. The “a(−)RptLig 3 prime” oligonucleotide aligns the 3 prime end of the RptLigMidCy3 and the 5 prime phosphorylated end of the dendrimer arm such that ligation will occur between the dendrimer arm and only one RptLigMidCy3 oligonucleotide.

Note that “a(−)RptLig 3 Prime” and “c(−)RptLig 5 Prime” should not be used on the same dendrimer, as this may cause a polymer to “loop” ligate from the 3 prime end of one arm to the 5 prime end of an adjacent arm of another type on the same dendrimer.

5′- gAC CCg TAA gAT AT -3′(SEQ. ID NO.2)

the “RptLigMidBridge” bridging oligonucleotide required for polymerizing the RptLigMidCy3 oligonucleotide is also required (previously described).

A typical ligation reaction is described below:
A. For the initial “priming” ligation, combine in a nuclease free polypropylene microfuge tube the following components:

$$\begin{matrix}100.0 uL - RptLigMidCy3 oligonucleotide at 1.0 ugms \text{/} uL ( aqueous ) \\ 100.0 uL - “ a ( - ) RptLig 3 prime ” Bridge oligonucleotide at 1.0 ugms \text{/} uL \\ ( aqueous ) OR “ c ( - ) RptLig 5 prime ” Bridge oligonucleotide \\ at 1.0 ugms \text{/} uL ( aqueous ) ( do not add both simultaneously ) \\ 20.0 uL - 4 layer plus core dendrimer 1.0 ugms \text{/} uL ( aqueous ) \\ 100.0 uL - 10 X Ligation Buffer ( Roche catalog number 1 \; 243 \; 292 ) , \\ 680.0 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1 U per uL, Roche catalog number 481 220). Mix gently with a pipetter.
F. Incubate at 4-20 C for 30-60 minutes (or longer).
G. Proceed to the polymerization ligation below.

Polymerization of RptLigMidCy3 to dendrimer pre-primed with a single RptLigMidCy3 per dendrimer arm:
H. To continue with the polymerization of RptLigMidCy3 to dendrimer pre-primed with a single RptLigMidCy3 per dendrimer arm, combine in a nuclease free polypropylene microfuge tube the following components:

$$\begin{matrix}1000.0 uL - Primed dendrimer \text{-} RptLigMidCy3 ligation reaction from \\ above \\ 50.0 uL - RptLigMidCy3 oligonucleotide at 1.0 ugms \text{/} uL ( aqueous ) \\ 130.0 uL - RptLigMidBridge Bridge oligonucleotide at 1.0 ugms \text{/} uL \\ ( aqueous ) \\ 20.0 uL - 10 X Ligation Buffer ( Roche catalog number 1 \; 243 \; 292 ) \\ 1 , 200.0 uL total volume\end{matrix}$$
I. Centrifuge the contents at 13,000 RPM in a microfuge centrifuge to bring all liquid to the bottom of the tube.
J. Incubate at 42 C for 15 minutes.
K. Cool to room temperature.
L. Add 20 uL of T4 DNA Ligase (1 U per uL, Roche catalog number 481 220). Mix gently with a pipetter.
M. Incubate at 4-20 C for 30-60 minutes (or longer).
N. Repeat steps H-M three (3) to six (6) more times to add the polymerization components to the reaction in additional cycles. Further cycles can, likewise, be added as many times as desired, to achieve further lengths of polymerization). Alternatively, the dendrimer molecules may be separated from the non-ligated reactants through the use of standard separation techniques (magnetic bead, affinity or size exclusion chromotography, membrane filtration, etc.) between or prior to “cycling” of the polymerization ligation reactions.
O. Stop the reaction by adding one-fortieth of the total reaction volume as 0.5M NaOH (aqueous).
P. Concentrate the volume to ˜100 uL using the Microcon YM-30 Microconcentrators (Millipore) according to the manufacturer's instructions.
   Typically, this method results in the ligation of between 1 and several hundreds or thousands of labeled polymeric oligonucleotides to each of the dendrimer's “arms”.

### EXAMPLE 5

### Labeling of a DNA Dendrimer with a Fluorescent Polymer by Ligation of Non-Specific Pre-Formed Polymer to the Dendritic Structure

Labeling of the DNA dendrimer with pre-formed polymeric molecules is performed similarly to the priming reaction of the above example except for the use of a multi-labeled polymeric molecule rather than the use of a non-polymeric labeled oligonucleotide. This method, which utilizes a simpler and faster procedure than Example 4 above, requires a supply of pre-ligated polymer as described in Example 1. Long pre-formed multi-labeled polymers are directly ligated to the “arms” of a DNA dendrimer through the use of a bridging oligonucleotide that simultaneously hybridizes to the “arms” of the dendrimer and to the appropriate ends of the multi-labeled polymer.

A typical ligation reaction is described below:
A. For the initial “priming” ligation, combine in a nuclease free polypropylene microfuge tube the following components:

$$\begin{matrix}100.0 uL - RptLigMidCy3 pre \text{-} formed multi \text{-} labeled polymer , average \\ size 100 –1000 nucleotides , at 1.0 ug ms \text{/} uL ( aqueous ) \\ 100.0 uL - “ a ( - ) RptLig 3 prime ” Bridge oligonucleotide at 1.0 ugms \text{/} uL \\ ( aqueous ) OR “ c ( - ) RptLig 5 prime ” Bridge oligonucleotide \\ at 1.0 ugms \text{/} uL ( aqueous ) ( do not add both simultaneously ) \\ 20.0 uL - 4 layer plus core dendrimer 1.0 ugms \text{/} uL ( aqueous ) \\ 100.0 uL - 10 X Ligation Buffer ( Roche catalog number 1 \; 243 \; 292 ) , \\ 680.0 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1 U per uL, Roche catalog number 481 220). Mix gently with a pipetter.
F. Incubate at 4-20 C for 60-240 minutes (or longer).
G. Stop the reaction by adding 40 uL of 0.5M NaOH (aqueous).
H. Concentrate the volume to 100 uL using the Microcon YM-30 Microconcentrators (Millipore) according to the manufacturer's instructions.
I. Purify the dendrimer molecules from smaller molecular species according to established methods.
   Typically, this method results in the ligation of between five (5) and several hundreds or thousands of labeled polymeric oligonucleotides to each of the dendrimer's “arms”.
   Note that “a(−)RptLig 3 Prime” and “c(−)RptLig 5 Prime” should not be used on the same dendrimer, as this may cause a polymer to loop ligate from one arm to an adjacent arm.

### EXAMPLE 6

### Labeling of a DNA Dendrimer with a Fluorescent Polymer by Ligation of a Sequence Specific Terminated Pre-Formed Polymer to the Dendritic Structure

Labeling of the DNA dendrimer with pre-formed polymeric molecules terminated with a sequence specific oligonucleotide is performed similarly to the priming reaction of the above example except for the use of a multi-labeled polymeric molecule containing a non-polymeric terminating sequence capable of hybridization to a complementary DNA molecule. This method is similar to Example 5 above except that bridging oligonucleotides complementary to the non-polymeric terminating sequence on the multi-labeled polymer must be used to bridge the polymeric molecule to the dendrimer arms. A bridge similar in function and design to “a(−)RptLig 3 Prime” or “c(−)RptLig 5 Prime” is required, with a different seven base 3 prime end sequence for “a(−)RptLig 3Prime” or seven base 5 prime end sequence for “c(−)RptLig 5 Prime” complementary to the appropriate seven (7) nucleotides of the terminating sequence on the multi-labeled polymer. Otherwise, the ligation reaction is performed similarly to the procedure in Example 5.

### EXAMPLE 7

### Labeling of a DNA Dendrimer with a Fluorescent Polymer by UV Crosslinking of a Sequence Specific Terminated Pre-Formed Polymer to the Dendritic Structure

Labeling of the DNA dendrimer with pre-formed polymeric molecules terminated with a sequence specific oligonucleotide may also be accomplished through the crosslinking of complementary DNA sequences through the use of ultraviolet light (UV) activated intercalators. Compounds such as 4,5,8-trimethylpsoralen are routinely used to irreversibly and covalently bind hybridized double stranded regions of DNA.

The crosslinking of pre-formed multi-labeled polymers to dendrimer branches or “arms” through the use of terminating sequences complementary to the sequences on or attached to the dendrimer “arms” results in the covalent attachment of multi-labeled polymeric molecules to the dendrimer.

A typical crosslinking reaction is:
A. Combine in a microfuge tube that is UV transparent:

$$\begin{matrix}100.0 uL - RptLigMidCy3 pre \text{-} formed multi \text{-} labeled polymer , \\ terminated with an appropriate oligonucleotide complemetary \\ to the dendrimer “ arms ” , average size 100 –1000 nucleotides , \\ at 1.0 ugms \text{/} uL ( aqueous ) \\ 20.0 uL - 4 layer plus core dendrimer 1.0 ugms \text{/} uL ( aqueous ) \\ 20.0 uL - 0.5 M NaCl ( aqueous ) \\ 75.0 uL - Ethanol saturated with 4 , 5 , 8 \text{-} trimethylpsoralen , room \\ temperature ( 20 –25 C . ) \\ 285.0 uL - Nuclease Free Water \\ 500 ul total volume\end{matrix}$$
B. Incubate at 75 C for 10 minutes.

C. Let slow cool to 35-42 C.

D. Expose to 1,000-10,000 uJoules (microjoules) of UV-A ultraviolet light.

E. Repeat addition of 4,5,8-trimethylpsoralen and exposure to UV-A.

F. Purify the dendrimer molecules from smaller species according to established methods.

### EXAMPLE 9

### Synthesis of a Polymer Containing Multiple Sequences Capable of Hybridizing a Secondary Labeled Nucleic Acid Molecule

Synthesis of a repeating polymeric DNA molecule containing multiple capture sequences that are complementary to a second fluorescent dye labeled molecule is easily accomplished by using a 46mer synthetic DNA oligonucleotide (synthetized by standard amidite chemistry methods) as the polymerizing component:

(SEQ. ID NO.11)

5′- phos - ACg ggT CCT CCA CTA CCg TCT Tgg TTT CAC
ATA CgA CTC ATA TCT T -3′

where the 5 prime end of the oligonucleotide is chemically (or enzymatically) phosphorylated. This oligonucleotide is called “a(−)RptLig”.

In addition, a bridging oligonucleotide capable of spanning the seven 5 prime nucleotides and seven 3 prime nucleotides of a(−)RptLig was synthesized utilizing standard amidite oligonucleotide synthesis chemistry, with the following sequence:

5′- gAC CCg TAA gAT AT -3′(SEQ. ID NO.2)

A typical ligation reaction is described below:
A. Combine in a nuclease free polypropylene microfuge tube the following components:

$$\begin{matrix}321.3 uL - a ( - ) RptLig oligonucleotide at 1.0 ugms \text{/} uL ( aqueous ) \\ 280.0 uL - RptLigMidBridge oligonucleotide at 1.0 ugms \text{/} uL ( aqueous ) \\ 100.0 uL - 10 X Ligation Buffer ( Roche catalog number 1 \; 243 \; 292 ) , \\ 298.7 uL - Nuclease Free Water \\ 1 , 000.0 uL total volume\end{matrix}$$
B. Centrifuge the contents at 13,000 RPM in a microfuge centrifuge to bring all liquid to the bottom of the tube.
C. Incubate at 42 C for 15 minutes.
D. Cool to room temperature.
E. Add 100 uL of T4 DNA Ligase (1U per uL, Roche catalog number 481 220). Mix gently with a pipetter.
F. Incubate at 4-20 C for 30-720 minutes (or longer). Longer incubation at temperatures below 20 C produce slightly more efficient ligation, resulting in somewhat longer concatenation ligation products.
G. Stop the reaction by adding 27.5 uL of 0.5M EDTA (aqueous).
H. Concentrate the volume to ˜100 uL using the Microcon YM-30 Microconcentrators (Millipore) according to the manufacturer's instructions.

The typical result of the above process is a heterogeneous range of sizes of the ligated a(−) RptLig polymeric molecules derived from single oligonucleotides. Purification of specific size ranges of polymers is accomplished as previously described in Example 1.

Repeating polymeric molecules may also be labeled with a terminating oligonucleotide in a process similar to that used in Examples 2 or 3. Terminated or non-terminated polymers containing repeats of a(−) RptLig may then be used in detection assays as a device for delivering multiple labeled oligonucleotides capable of hybridizing to the a(−)RptLig sequence, containing the complementary sequence labeled on the 5 prime end with a Cy3 fluorescent dye:

(SEQ. ID NO.12)

5′- Cy3 - gAg TCg TAT gTg AAA CCA AgA Cgg TAg Tgg
A -3′

Having described the invention, it will be understood that the foregoing discussion discloses and describes merely exemplary and preferred embodiments of the present invention. One skilled in the art will readily recognize from such discussion, and from the accompanying drawings and examples, that various changes, modifications and variations can be made therein without departing from the spirit and scope of the inventions as defined in the claims. One skilled in the art may likewise by applying current or future knowledge, adopt the same for use in accordance with the present invention. Yet, having described this invention with regard to specific embodiments, it is to be understood that the description is not meant as a limitation, and that the present application covers all such embodiments, modifications and variations of the presently disclosed inventions.

1. A method comprising the steps of:
(a) providing a population of oligonucleotides comprising labeling monomers, wherein said labeling monomers are oligonucleotides of the structure 5′-AB-3′, wherein AB is a nucleotide sequence such that A corresponds to at least one nucleotide and B corresponds to at least one nucleotide; wherein at least one nucleotide in said nucleotide sequence AB incorporates a label or is provided for having a label bound thereto; said labeling monomers comprising a 5 prime phosphate or being capable of being 5 prime phosphorylated, said labeling monomers further comprising a 3 prime hydroxyl or being capable of receiving a 3 prime hydroxyl; and,
(b) synthesizing a polymer for use as a label molecule, said polymer being a polymer synthesized from said labeling monomers, said polymer comprising a sequence of said oligonucleotides, wherein said sequence of said oligonucleotides comprises repeats of said sequence AB.

2. A method as claimed in claim 1, wherein said polymer is synthesized by ligation of said oligonucleotides.

3. (canceled)

4. A method as claimed in claim 1, wherein said labeling monomers are labeled monomers.

5-7. (canceled)

8. A method as claimed in claim 1, wherein said population comprises at least two sub-populations of labeling molecules.

9. A method as claimed in claim 1, wherein said population comprises at least three sub-populations of labeling molecules.

10. A method as claimed in claim 1, further comprising the step of adding an oligonucleotide to the 5 prime end of said linear polymer, said oligonucleotide being added to serve as a targeting oligonucleotide.

11. A method as claimed in claim 1, further comprising the step of adding an oligonucleotide to the 3 prime end of said linear polymer, said oligonucleotide being added to serve as a targeting oligonucleotide.

12. A method as claimed claim 1, further comprising the step of attaching said polymer to a delivery device.

13. (canceled)

14. A method comprising the steps of:
(a) providing a first population of oligonucleotides, said first population comprising labeling monomers, wherein said labeling monomers are oligonucleotides of the structure 5′-AB-3′, wherein AB is a nucleotide sequence such that A corresponds to at least one nucleotide and B corresponds to at least one nucleotide; wherein at least one nucleotide in said nucleotide sequence AB incorporates a label or is provided for having a label bound thereto; said labeling monomers comprising a 5 prime phosphate or being capable of being 5 prime phosphorylated, said labeling monomers further comprising a 3 prime hydroxyl or being capable of receiving a 3 prime hydroxyl;
(b) providing a second population of oligonucleotides, said second population comprising bridging oligonucleotides, said bridging oligonucleotides being oligonucleotides having nucleic acid sequences complementary to said labeling monomers, wherein the sequence of each bridging oligonucleotide is provided such that said bridging oligonucleotides can each bind at least two of said labeling monomers;
(c) mixing said first population and said second population to allow said bridging nucleotides to bind said labeling monomers and align said labeling monomers m a configuration suitable for ligation of said label monomers said oligonucleotides of said first population; and,
(d) ligating said oligonucleotides of said first population to form a polymer for use as a label molecule, said polymer being a linear polymer synthesized from said labeling monomers, said linear polymer comprising a sequence of said oligonucleotides, wherein said sequence of said oligonucleotides comprises repeats of said sequence AB.

15-16. (canceled)

17. A method as claimed in claim 14, wherein said labeling monomers are labeled monomers.

18-20. (canceled)

21. A method as claimed in claim 14, wherein said population comprises at least two sub-populations of labeling molecules.

22. A method as claimed in claim 14, wherein said population comprises at least three sub-populations of labeling molecules.

23. A method as claimed in claim 14, further comprising the step of adding an oligonucleotide to the 5 prime end of said linear polymer.

24. A method as claimed in claim 14, further comprising the step of adding an oligonucleotide to the 3 prime end of said linear polymer, said oligonucleotide being added to serve as a targeting oligonucleotide.

25. A method as claimed in claim 23, wherein said oligonucleotide is added to serve as a targeting oligonucleotide.

26. A method as claimed in claim 14, further comprising the step of attaching said polymer to a delivery device.

27. (canceled)

28. A method comprising the steps of:
(a) providing a first population of oligonucleotides, said first population comprising labeling monomers, wherein said labeling monomers are oligonucleotides of the structure 5′AB-3′, wherein AB is a nucleotide sequence such that A corresponds to at least one nucleotide and B corresponds to at least one nucleotide; wherein at least one nucleotide in said nucleotide sequence AB incorporates a label or is provided for having a label bound thereto; said labeling monomers comprising a 5 prime phosphate or being capable of being 5 prime phosphorylated, said labeling monomers further comprising a 3 prime hydroxyl or being capable of receiving a 3 prime hydroxyl;
(b) providing a second population of oligonucleotides, said second population comprising bridging oligonucleotides, said bridging oligonucleotides being oligonucleotides having nucleic acid sequences complementary to said labeling monomers, wherein the sequence of each bridging oligonucleotide is provided such that said bridging oligonucleotides can each bind at least two of said labeling monomers;
(c) mixing said first population and said second population to allow said bridging nucleotides to bind said labeling monomers and align said labeling monomers in a configuration suitable for ligation of said label monomers said oligonucleotides of said first population; and,
(d) ligating said oligonucleotides of said first population to form a polymer for use as a label molecule, said polymer being a linear polymer synthesized from said labeling monomers, said linear polymer comprising a sequence of said oligonucleotides, wherein said sequence of said oligonucleotides comprises repeats of said sequence AB; and,
(e) adding a terminating oligonucleotide to at least one of the ends of said linear polymer.

29-36. (canceled)

37. A method as claimed in claim 28, further comprising the step of adding an oligonucleotide to the 5 prime end of said linear polymer, said oligonucleotide being added to serve as a targeting oligonucleotide.

38. A method as claimed in claim 28, further comprising the step of adding an oligonucleotide to the 3 prime end of said linear polymer, said oligonucleotide being added to serve as a targeting oligonucleotide.

39. (canceled)

40. A method as claimed in claim 28, further comprising the step of attaching said polymer to a delivery device.

41-47. (canceled)
